(19) United States
(12) Patent Application Publication
(10) Pub. No.: US 2026/0083729 A1
(43) Pub. Date: Mar. 26, 2026

(54) METHODS FOR TREATMENT OF BONE MARROW FAILURE AND RELATED CONDITIONS

(71) Applicant: Imago Biosciences, Inc., San Carlos, CA (US)

(72) Inventors: Maria Kleppe, New York, NY (US); Hugh Y. Rienhoff, JR., San Carlos, CA (US); Jennifer Dias, Dublin, CA (US)

(73) Assignee: Imago Biosciences, Inc., San Carlos, CA (US)

(21) Appl. No.: 19/137,870

(22) PCT Filed: Dec. 15, 2023
(86) PCT No.: PCT/US2023/084159

§ 371 Date: Jun. 11, 2025

Related U.S. Application Data
(60) Provisional application No. 63/477,020, filed on Dec. 23, 2022.

(51) Int. Cl. A61K 31/496; A61K 35/12; A61P 7/00

(52) U.S. Cl. CPC A61K 31/496; A61K 35/12; A61P 7/00; A61K 2035/124

(57) ABSTRACT

Provided herein are methods for increasing the numbers of hematopoietic stem cells (HSCs) in a patient in need thereof, for treating a condition characterized by reduced numbers of hematopoietic stem cells (HSCs), such as bone marrow failure (BMF) or a BMF syndrome, in a patient in need thereof, for enhancing recovery from or improving tolerance of a near-ablative cancer therapy, and/or for reducing the damage to, or accelerating the recovery of, the hematopoietic compartment after a physical insult thereto in a patient in need thereof, comprising administering in vivo or treating cells ex vivo with an LSD1 inhibitor.

### CROSS-REFERENCE TO RELATED APPLICATIONS

This international application claims the benefit of priority to U.S. Provisional Application No. 63/477,020, filed Dec. 23, 2022, the entirety of which is incorporated herein by reference.

### FIELD OF INVENTION

Bone marrow is the primary site of production for blood cells, including red blood cells, which carry oxygen throughout the body via the protein hemoglobin, white blood cells, which fight infection, and platelets, which facilitate blood clotting. All blood cells arise from a common progenitor called the hematopoietic stem cell (HSC).

Bone marrow failure (BMF) describes a clinical situation in which the blood-forming portion of bones fails to produce sufficient blood cells of one or more hematopoietic lineages, leading to a loss of functional blood cells and attendant cytopenias. All BMF syndromes ultimately arise because of an intrinsic defect in or an insult to the HSC including insults or injury to the cellular niche that supports the maintenance of HSCs. Intrinsic defects include mutations that impair normal HSC function, while extrinsic factors include any pathological alteration in the bone marrow niche, the microenvironment that supports the HSC, that impairs normal functioning of the HSC as well as toxic insults or immune destruction of the HSC itself. Extrinsic factors outside the bone marrow can include autoimmune antibodies or immune cells that target HSCs. the bone marrow itself being altered by a pathologic process, such as leukemia or fibrosis, that renders the bone marrow niche inadequate for HSC survival, exposure of the bone marrow to an insult such as radiation, chemotherapy, or chemical toxins that kill HSCs, or depletion of HSCs secondary to intrinsic—often genetic—defects that ultimately lead to their premature death.

Depending on the cause, BMF and other conditions characterized by reduced numbers of HSCs can manifest at any age, with inherited conditions generally presenting early in life, autoimmune causes in early adulthood, and sporadic conditions typically later in life. The incidence of inherited BMFs accounts for 10% to 15% of marrow aplasia and 30% of pediatric BMF disorders, with approximately 65 cases per million live births every year. The most common inherited BMF syndrome is Fanconi anemia (FA) which occurs in 1 to 5 cases per million with a carrier frequency of 1 in 200-300, although it is more common in certain ethnic groups. e.g., Spanish Roma (1 in 64), Afrikaners in South Africa with a specific genetic mutation (1 in 83), and Ashkenazi Jews (1 in 89).

Many treatments for BMF syndromes and other conditions characterized by reduced numbers of HSCs are known in the art. Supportive care, including transfusions of red blood cells or platelets, are used to reduce signs and symptoms of disease. Growth factors that stimulate the proliferation of specific lineages, such as erythropoietin or androgens for enhancing red cell production, thrombopoietics for platelet production, and/or granulocyte colony-stimulating factors for neutrophil production can be administered. However, these treatments do not provide a cure, nor do they specifically enhance the population of HSCs. Hematopoietic stem cell transplantation (HSCT), i.e., bone marrow transplantation, can be curative and involves the infusion of healthy blood stem cells derived from a matched donor into the patient to re-populate the bone marrow with HSCs that then can restore the production of blood cells. However, the availability of such a transplant, e.g., from a matched donor, can be limited, especially for patients with rare genotypes or underrepresented ethnicities in bone marrow repositories, and especially in the time frame during which the transplant is needed. Additionally, patients undergoing HSCT can be severely immunocompromised and require intense supportive treatment, especially in the several weeks or months following transplant. Mortality is high. In cases of autoimmune destruction of HSCs, immunosuppression may be used for patients who are not eligible for HSCT or for whom a suitable donor is not available. Thus, safer, less invasive, and less expensive treatments for BMF are needed.

Accordingly, provided herein are methods of
increasing the numbers of hematopoietic stem cells (HSCs) in a patient in need thereof,
treatment of a condition characterized by reduced numbers of hematopoietic stem cells (HSCs) in a patient in need thereof,
treating a condition characterized by reduced numbers of hematopoietic stem cells (HSCs) in a patient in need thereof,
enhancing recovery from or improving tolerance of a near-ablative cancer therapy, and
reducing the damage to, or accelerating the recovery of, the hematopoietic compartment after a physical insult thereto in a patient in need thereof,
   comprising administering an LSD1 inhibitor to the patient in an amount and/or for a period of time sufficient to result in an in vivo expansion of a population of HSCs, or contacting HSCs harvested from a patient or a suitable donor with an LSD1 inhibitor ex vivo, in an amount and/or for a period of time sufficient to result in an expansion of a population of HSCs, then administering to the patient a therapeutically effective amount of the expanded population of HSCs.

These and other embodiments disclosed herein are described in detail below.

### BRIEF DESCRIPTION OF THE DRAWINGS

FIG. 1—Shows the in vivo expansion of LSK cells (HSC/progenitor cells: Lineage⁻, Sca-1⁺, c-Kit⁺) by LSD1 inhibitors IMG-7289 (15 mg/kg: 2-fold increase (966:2545); 40 mg/kg: 6-fold increase) and Compound 2 (40 mg/kg: 1.7-fold increase (1033:1714); 80 mg/kg: 2.3-fold increase (917:2215)).

FIG. 2—Shows the in vivo expansion of long-term HSC (LT-HSC) cells by LSD1 inhibitors IMG-7289 (15 mg/kg: 2-fold increase (120:252); 40 mg/kg: 7.5-fold increase (120:921)) and Compound 2 (40 mg/kg: 1.5-fold increase (165:250); 80 mg/kg: 1.6-fold increase (96:157)).

FIG. 3—Shows the effect of IMG-7289 on complete blood count as evidenced by platelet (PLT) levels at two concentrations; CBC is reduced in a dose-dependent manner.

FIG. 4—Shows the effect of Compound 2 on complete blood count as evidenced by platelet (PLT) levels at two concentrations; CBC is not significantly affected at either dose.

FIG. 5—Shows the effect of Compound 2 on complete blood count as evidenced by platelet (PLT) levels at three higher concentrations; CBC is not significantly affected at any dose.

FIG. 6—Shows the effect of DMSO (negative control), LSD1-CoREST-HDAC complex degrader UM-171, and LSD1 inhibitors IMG-7289 and Compound 2 on the total number of CD34+EPCR+ cells per well.

FIG. 7—Shows the effect of DMSO (negative control), LSD1-CoREST-HDAC complex degrader UM-171, and LSD1 inhibitors IMG-7289 and Compound 2 on percent CD34+EPCR+ cells per well.

FIG. 8—Shows the effect of DMSO (negative control), LSD1-CoREST-HDAC complex degrader UM-171, and LSD1 inhibitors IMG-7289 and Compound 2 on the total number of CD34+CD38−CD45RA-EPCR+ cells per well.

FIG. 9—Shows the effect of DMSO (negative control), LSD1-CoREST-HDAC complex degrader UM-171, and LSD1 inhibitors IMG-7289 and Compound 2 on percent CD34+CD38−CD45RA-EPCR+ cells per well.

FIG. 10—Shows percent increase in CD34+EPCR+CD38−CD45RA− HSCs as a result of treatment with DMSO (control); EPCR is on the vertical axis and CD38 on the horizontal.

FIG. 11—Shows percent increase in CD34+EPCR+CD38−CD45RA− HSCs as a result of treatment with LSD1-CoREST-HDAC complex degrader UM-171; EPCR is on the vertical axis and CD38 on the horizontal.

FIG. 12—Shows percent increase in CD34+EPCR+CD38−CD45RA− HSCs as a result of treatment with 100 nm LSD1 inhibitor Compound 2: EPCR is on the vertical axis and CD38 on the horizontal.

FIG. 13—Shows percent increase in CD34+EPCR+CD38−CD45RA− HSCs as a result of treatment with 30 nm LSD1 inhibitor IMG-7289; EPCR is on the vertical axis and CD38 on the horizontal.

FIG. 14—Shows percent increase in CD34+EPCR+CD38−CD45RA− HSCs as a result of treatment with 10 nm LSD1 inhibitor Compound 1: EPCR is on the vertical axis and CD38 on the horizontal.

FIG. 15—Shows percent increase in CD34+EPCR+CD38−CD45RA− HSCs as a result of treatment with 30 nm LSD1 inhibitor Compound 1; EPCR is on the vertical axis and CD38 on the horizontal.

FIG. 16—Shows the effect of DMSO (negative control), LSD1-CoREST-HDAC complex degrader UM-171, and LSD1 inhibitors Compound 1, Compound 2, IMG-7289, and Compound 4 on the total number of CD34+ cells per well.

FIG. 17—Shows the effect of DMSO (negative control), LSD1-CoREST-HDAC complex degrader UM-171, and LSD1 inhibitors Compound 1, Compound 2, IMG-7289, and Compound 4 on the total number of CD34+EPCR+ cells per well.

FIG. 18—Shows the effect of DMSO (negative control), LSD1-CoREST-HDAC complex degrader UM-171, and LSD1 inhibitors Compound 1, Compound 2, IMG-7289, and Compound 4 on the total number of CD34+CD38−CD45RA-EPCR+ cells per well.

FIG. 19—Shows the effect of DMSO (negative control), LSD1-CoREST-HDAC complex degrader UM-171, and LSD1 inhibitors Compound 1, Compound 2, IMG-7289, and Compound 4 on the total number of CD34+CD38−CD90+EPCR+ cells per well.

### DETAILED DESCRIPTION

Provided herein is a method of expanding or increasing the numbers of hematopoietic stem cells (HSCs) in vivo in a patient having a condition characterized by reduced numbers of HSCs comprising administration of an LSD1 inhibitor to the patient. In some embodiments, a method of the present disclosure enhances the capacity of bone marrow to produce all lineages of blood cells in a subject suffering from a BMF syndrome. In some embodiments, a method of the present disclosure enhances the capacity of the bone marrow to resist an insult. In some embodiments, a method described herein comprises: (i) administering an LSD1 inhibitor to a patient for a period of time; wherein the administration of the LSD1 inhibitor results in the in vivo expansion of a population of bone marrow HSCs; or (ii) administering to said patient a therapeutically effective amount of bone marrow HSCs, wherein the bone marrow HSCs are treated ex vivo with an LSD1 inhibitor for a period of time prior to hematopoietic stem cell transplantation, resulting in the expansion of the bone marrow HSCs.

The invention is further illustrated by the following embodiments.

Provided herein is a method of increasing the numbers of hematopoietic stem cells (HSCs) in a patient in need thereof comprising i) administering an LSD1 inhibitor to the patient in an amount and/or for a period of time sufficient to result in an in vivo expansion of a population of HSCs; or ii) contacting HSCs harvested from a patient or a suitable donor with an LSD1 inhibitor ex vivo, in an amount and/or for a period of time sufficient to result in an expansion of a population of HSCs, then administering to the patient a therapeutically effective amount of the expanded population of HSCs.

In some embodiments, the patient has a condition characterized by reduced numbers of HSCs.

Also provided herein is a method of treating a condition characterized by reduced numbers of hematopoietic stem cells (HSCs) in a patient in need thereof, comprising i) administering an LSD1 inhibitor to the patient in an amount and/or for a period of time sufficient to result in an in vivo expansion of a population of HSCs; or ii) contacting HSCs harvested from a patient or a suitable donor with an LSD1 inhibitor ex vivo, in an amount and/or for a period of time sufficient to result in an expansion of a population of HSCs, then administering to the patient a therapeutically effective amount of the expanded population of HSCs.

Also provided herein is a method of enhancing recovery from a near-ablative cancer therapy comprising, subsequent to such therapy, i) administering an LSD1 inhibitor to the patient in an amount and/or for a period of time sufficient to result in an in vivo expansion of a population of hematopoietic stem cells (HSCs); or ii) contacting HSCs harvested from a patient or a suitable donor with an LSD1 inhibitor ex vivo, in an amount and/or for a period of time sufficient to result in an expansion of a population of HSCs, then administering to the patient a therapeutically effective amount of the expanded population of HSCs.

Also provided herein is a method of improving tolerance of a near-ablative cancer therapy comprising, prior to such therapy, i) administering an LSD1 inhibitor to the patient in an amount and/or for a period of time sufficient to result in an in vivo expansion of a population of hematopoietic stem cells (HSCs); or ii) contacting HSCs harvested from a patient or a suitable donor with an LSD1 inhibitor ex vivo, in an amount and/or for a period of time sufficient to result in an expansion of a population of HSCs, then administering to the patient a therapeutically effective amount of the expanded population of HSCs.

In some embodiments, the near-ablative cancer therapy is administered after a period of time after the administration of the LSD1 inhibitor sufficient for cells to re-enter the quiescent phase.

In some embodiments, the period of time sufficient for cells to enter the quiescent phase is between about one and about two weeks.

In some embodiments, the near-ablative cancer therapy is chosen from therapeutic irradiation of cancer cells and cytotoxic chemotherapy of cancer cells.

Also provided herein is a method of reducing the damage to, or accelerating the recovery of, the hematopoietic compartment after a physical insult thereto in a patient in need thereof, comprising i) administering an LSD1 inhibitor to the patient in an amount and/or for a period of time sufficient to result in an in vivo expansion of a population of HSCs; or ii) contacting HSCs harvested from a patient or a suitable donor with an LSD1 inhibitor ex vivo, in an amount and/or for a period of time sufficient to result in an expansion of a population of HSCs, then administering to the patient a therapeutically effective amount of the expanded population of HSCs.

In some embodiments, the physical insult is a result of irradiation or chemical toxicity.

In some embodiments, administration of the LSD1 inhibitor results in proliferation of the hematopoietic stem cells in the bone marrow.

In some embodiments, administration of the LSD1 inhibitor reduces a proportion of quiescent (Go) cells in the hematopoietic stem cell population.

In some embodiments, administration of the LSD1 inhibitor results in the hematopoietic stem cells entering the cell division cycle, thereby increasing the number of hematopoietic stem cells in the bone marrow and in the peripheral circulation.

In some embodiments, the HSCs are capable of self-renewal (i.e., are not progenitor cells or HSPCs).

In some embodiments, the HSCs are long-term HSCs (LT-HSCs).

In some embodiments, the HSCs are short-term HSCs (ST-HSCs).

In some embodiments, the HSCs express EPCR.

In some embodiments, the HSCs express CD34 and EPCR but not CD45RA or CD38.

In some embodiments, the LT-HSCs express any or all of CD90, CD49f, EPCR, and/or CD34, but not CD38 and/or CD45RA.

In some embodiments, the numbers of HSCs are reduced by 50% prior to administration of the LSD1 inhibitor.

In some embodiments, the reduced numbers of HSCs classify the patient as having bone marrow failure (BMF).

In some embodiments, reduced numbers of peripheral blood cells, i.e., cytopenia, anemia, thrombocytopenia, lymphopenia, or neutropenia classify the patient as having bone marrow failure (BMF).

In some embodiments, the condition characterized by reduced numbers of HSCs is chosen from a bone marrow failure syndrome, a myeloproliferative neoplasm, and a myelodysplastic syndrome.

In some embodiments, the condition characterized by reduced numbers of HSCs is not myelofibrosis (MF).

In some embodiments, the condition characterized by reduced numbers of HSCs is a clonal disease arising from a single mutated cell.

In some embodiments, the condition characterized by reduced numbers of HSCs is the result of a mutation, either germline (inherited or sporadic) or arising from a somatic mutation.

In some embodiments, the condition characterized by reduced numbers of HSCs is caused by a somatic mutation.

In some embodiments, the somatic mutation occurs in a gene related to hormone signaling.

In some embodiments, the condition characterized by reduced numbers of HSCs is a bone marrow failure syndrome.

In some embodiments, the bone marrow failure syndrome is an inherited bone marrow failure syndrome.

In some embodiments, the inherited bone marrow failure syndrome is chosen from Fanconi anemia, dyskeratosis congenita and other telomere biology disorders, Shwachman-Diamond syndrome, Diamond-Blackfan anemia, aplastic anemia, reticular dysgenesis, a GATA2-related disorder, a SAMD9/SAMD9L-related disorder, congenital amegakaryocytic thrombocytopenia, ADA2 deficiency, MIRAGE (major findings of Myelodysplasia, Infection, Restriction of growth, Adrenal hypoplasia, Genital phenotypes, and Enteropathy) syndrome, Paroxysmal nocturnal hemoglobinuria (PNH), Pearson syndrome, thrombocytopenia with absent radii (TAR), and a congenital neutropenia (e.g., one associated with a gene chosen from ELANE, HAX1, G6PC3, GF11, CSF3R, a patient with X-linked WAS, CXCR4, VPS45A, and JAGN1).

In some embodiments, the inherited bone marrow failure syndrome is chosen from Fanconi anemia, dyskeratosis congenita, Shwachman-Diamond syndrome, congenital amegakaryocytic thrombocytopenia, Blackfan-Diamond anemia, reticular dysgenesis, a GATA2-related disorder, and a SAMD9/SAMD9L-related disorder.

In some embodiments, the BMF syndrome is Fanconi anemia or Blackfan-Diamond anemia.

In some embodiments, the condition characterized by reduced numbers of HSCs is an autoimmune condition causing aplastic anemia.

In some embodiments, the condition characterized by reduced numbers of HSCs is the result of a mutation in a lineage cell.

In some embodiments, the lineage cell is a progenitor cell of the erythroid lineage, megakaryocytic lineage, or granulo-monocytic lineage.

In some embodiments, the lineage cell is chosen from common myeloid progenitor (CMP), erythroid progenitor cell (ERP), megakaryocytic progenitor cell (MkP), a granulocyte-monocyte progenitor (GMP), or a megakaryocytic-erythroid progenitor cell (MEP).

In some embodiments, the mutation in a lineage cell leads to altered numbers of, malformations in, or impaired function of one or more cells chosen from granulocytes, megakaryocytes, erythrocytes, neutrophils, basophils, eosinophils, monocytes, and dendritic cells.

In some embodiments, the myeloproliferative neoplasm is chosen from polycythemia vera (PV) and essential thrombocytosis (ET), chronic neutrophilic leukemia (CNL), chronic eosinophilic leukemia (CEL), chronic myelogenous leukemia (CML), and mastocytosis.

In some embodiments, the myelodysplastic syndrome is chosen from myelomonocytic leukemia, juvenile myelomonocytic leukemia, atypical chronic myelogenous leukemia (CML).

In some embodiments, the bone marrow failure syndrome is a result of iatrogenic myeloablative therapy.

In some embodiments, the bone marrow failure syndrome is a result of irradiation or chemical toxicity to the bone marrow.

In some embodiments, the irradiation is sustained in the course of cancer therapy.

In some embodiments, the chemical is chosen from busulfan, 5-fluorouracil (5-FU), and chloramphenicol.

In some embodiments, the LSD1 inhibitor is administered for a period of about 1 days to about 7 days.

In some embodiments, the LSD1 inhibitor is chosen from a reversible inhibitor and an irreversible inhibitor.

In some embodiments, the LSD1 inhibitor is an irreversible inhibitor or a reversible inhibitor with moderate-to-high binding affinity to LSD1.

In some embodiments, the LSD1 inhibitor is chosen from ranylcypromine (TCP), ORY-1001 (iadademstat), GSK-2879552, Compound 3 (bomedemstat), Compound 1, Compound 2, Compound 4, INCB059872, and ORI-2001 (vafidemstat).

In some embodiments, the LSD1 inhibitor is chosen from Compound 1, Compound 2, Compound 4, and Compound 3 (bomedemstat).

In some embodiments, the dose of the LSD1 inhibitor is from about 0.25 mg/kg to about 7 mg/kg.

In some embodiments, the dose of the LSD1 inhibitor is from about 10 mg to about 700 mg.

In some embodiments, administration of the LSD1 inhibitor does not reduce the population of platelets in the patient to Grade 4 thrombocytopenia.

In some embodiments, Grade 4 thrombocytopenia is characterized by a platelet count of less than 25×10⁹/L.

In some embodiments, the period of time comprises one cycle.

In some embodiments, one cycle is from about 1 day to about 7 days.

In some embodiments, the period of time is about 5 days.

In some embodiments, the period of time is repeated for an additional 1 to 10 cycles.

In some embodiments, administration of the LSD1 inhibitor is terminated after the period of time.

In some embodiments, administration of the LSD1 inhibitor is continued after the period of time.

In some embodiments, the LSD1 inhibitor prevents removal of mono- and di-methyl groups on lysine (K) residues present in histone 3 (H3).

In some embodiments, the lysine residues comprise K4 residues on H3.

In some embodiments, the LSD1 inhibitor is chosen from:
(1) a compound of formula (I):

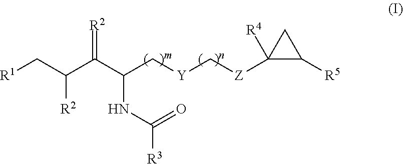

or a salt, polymorph, or solvate thereof, wherein:
Y is chosen from a bond, NR^{4a}, O, C(O)NH, NHC(O), S, SO₂, CHOH, and CH₂;
Z is chosen from a bond, NR^{4b}, O, C(O)NH, NHC(O), S, SO₂, and CH₂;
m is chosen from 0, 1, 2, 3, 4, and 5;
n is chosen from 0, 1, 2, and 3;
R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R³ is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups;
R⁴, R^{4a}, and R^{4b} are independently chosen from hydrogen, alkyl, alkenyl, alkynyl, and cycloalkyl;
R⁵ is chosen from aryl and heteroaryl, any of which may be optionally substituted with between 0 and 3 R⁶ groups;
each R⁶ is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl;
(2) a compound of formula (V)

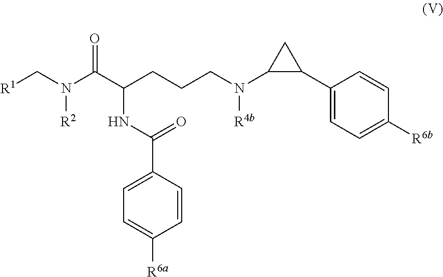

or a salt, polymorph, or solvate thereof, wherein:
R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
R^{4b} is chosen from hydrogen, alkyl, alkenyl, alkynyl, and cycloalkyl;
R^{6a} is chosen from heteroaryl, cyano, and S(O)₂N(CH₃)₂;
each R⁶ and each R^{6b} is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl;
(3) a compound of structural Formula (Ia):

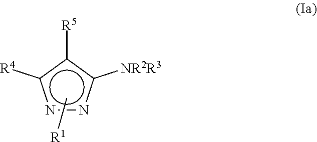

or a salt or tautomer thereof, wherein:
R¹ is chosen from cycloalkyl, heterocycloalkyl, aryl, and heteroaryl, any of which is optionally substituted with one R⁶, and one or more R⁷;
R² and R³ are independently chosen from H, cycloalkyl, heterocycloalkyl, aryl, heteroaryl, (cycloalkyl)alkyl, (heterocycloalkyl)alkyl, (aryl)alkyl, and (heteroaryl)alkyl, any of which is optionally substituted with one or more R⁸,
or R² and R³, together with the intervening nitrogen, combine to form heterocycloalkyl which is optionally substituted with one or more R⁸;
R⁴ is chosen from cycloalkyl, heterocycloalkyl, aryl, and heteroaryl, any of which is optionally substituted with one or more R⁹;
R⁵ is chosen from H, halo, and cyano;
R⁶ is chosen from cycloalkyl, heterocycloalkyl, aryl, heteroaryl, (cycloalkyl)carbonyl, (heterocycloalkyl)carbonyl, (aryl)carbonyl, and (heteroaryl)carbonyl, any of which is optionally substituted with one or more R¹⁰;
each R⁷ is independently chosen from halo, cyano, hydroxy, alkyl, and alkoxy;
each R⁸ is independently chosen from —NHR¹¹, —C(NH)NHR¹¹, —NHC(═NH)—R¹¹, —NHC(═NH)NHR¹¹, —CONHR¹¹, —CH₂NHR¹¹, —CH₂C(═NH)NHR¹¹, —CH₂NHC(═NH)—R¹¹, —CH₂NHC(═NH)NHR¹¹, —CH₂CONHR¹¹, halo, cyano, hydroxy, alkyl, and alkoxy;
each R⁹ is independently chosen from halo, cyano, hydroxy, alkyl, and alkoxy; and
each R¹⁰ is independently chosen from halo, cyano, hydroxy, oxo, alkyl, alkoxy, (hydroxy)alkyl. (hydroxy)alkoxy, alkylthio, and alkylsulfonyl; and
each R¹¹ is independently chosen from H and alkyl;
(4) a compound of structural Formula (IIa):

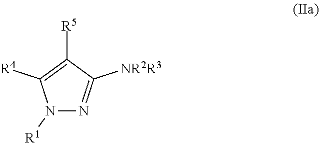

or a salt or tautomer thereof, wherein:
R¹ is chosen from cycloalkyl, heterocycloalkyl, aryl, and heteroaryl, any of which is optionally substituted with one R⁶, and one or more R⁷;
R² and R³ are independently chosen from H, cycloalkyl, heterocycloalkyl, aryl, heteroaryl, (cycloalkyl)alkyl, (heterocycloalkyl)alkyl, (aryl)alkyl, and (heteroaryl)alkyl, any of which is optionally substituted with one or more R⁸,
or R² and R³, together with the intervening nitrogen, combine to form heterocycloalkyl which is optionally substituted with one or more R⁸;
R⁴ is chosen from cycloalkyl, heterocycloalkyl, aryl, and heteroaryl, any of which is optionally substituted with one or more R⁹;
R⁵ is chosen from H, halo, and cyano;
R⁶ is chosen from cycloalkyl, heterocycloalkyl, aryl, heteroaryl, (cycloalkyl)carbonyl, (heterocycloalkyl)carbonyl, (aryl)carbonyl, and (heteroaryl)carbonyl, any of which is optionally substituted with one or more R¹⁰;
each R⁷ is independently chosen from halo, cyano, hydroxy, alkyl, and alkoxy;
each R⁸ is independently chosen from —NHR¹¹, —C(NH)NHR¹¹, —NHC(═NH)—R¹¹, —NHC(═NH)NHR¹¹, —CONHR¹¹, —CH₂NHR¹¹, —CH₂C(═NH)NHR¹¹, —CH₂NHC(═NH)—R¹¹, —CH₂NHC(═NH)NHR¹¹, —CH₂CONHR¹¹, halo, cyano, hydroxy, alkyl, and alkoxy;
each R⁹ is independently chosen from halo, cyano, hydroxy, alkyl, and alkoxy; and
each R¹⁰ is independently chosen from halo, cyano, hydroxy, oxo, alkyl, alkoxy, (hydroxy)alkyl, (hydroxy)alkoxy, alkylthio, and alkylsulfonyl; and
each R¹¹ is independently chosen from H and alkyl; and
(5) a compound of formula (Ib):

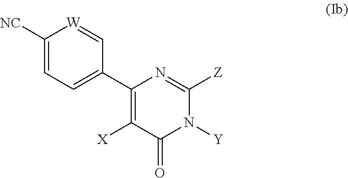

or a salt or tautomer thereof, wherein,
W is N, C—H, or C—F;
X is hydrogen, halogen, —CN, optionally substituted alkyl, optionally substituted alkynyl, optionally substituted carbocyclylalkynvl, optionally substituted aryl, or optionally substituted heteroaryl;
Y is hydrogen, optionally substituted alkyl, optionally substituted cycloalkyl, or optionally substituted cycloalkylalkyl;
Z is an optionally substituted group chosen from alkyl, carbocyclyl, C-attached heterocyclyl, N-attached heterocyclyl, heterocyclylalkyl, heterocyclylalkenyl, —O-heterocyclyl, —N(R)-heterocyclyl, —O-heterocyclylalkyl, —N(R)-heterocyclylalkyl, —N(R)(C₁-C₄alkylene)-NR₂, —O(C₁-C₄alkylene)-NR₂; and
R is hydrogen or C₁-C₄alkyl.

In some embodiments, the LSD1 inhibitor is chosen from:

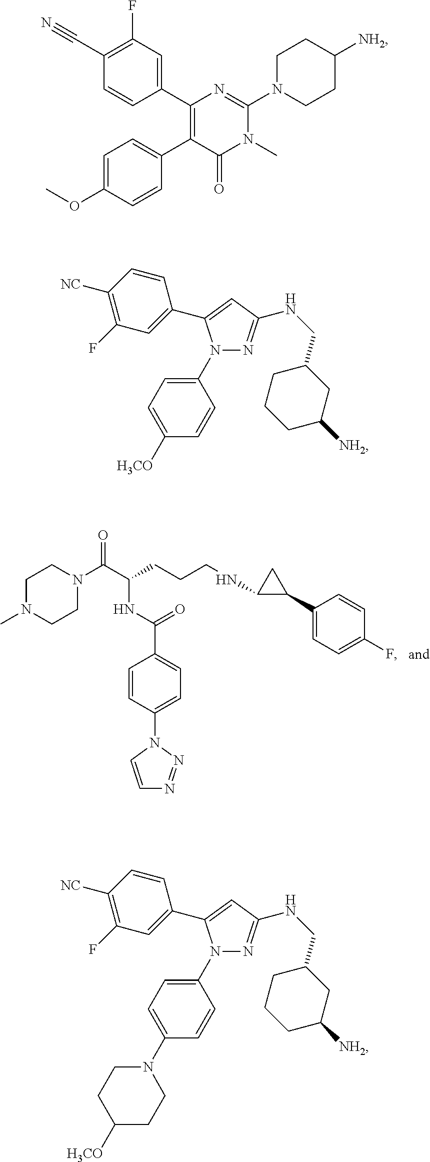

or a salt, polymorph, solvate, or tautomer thereof.

In some embodiments, the LSD1 inhibitor is a salt of the formula:

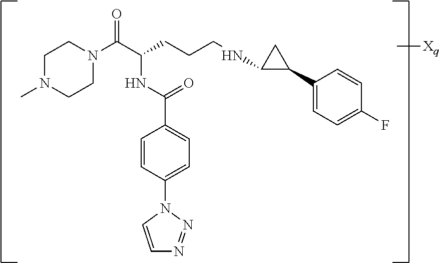

or a polymorph or solvate thereof, wherein:
X is chosen from tosylate, sulfate, tartrate, oxalate, besylate, fumarate, citric, esylate, and malate; and
q is an integer chosen from 1 and 2.

In some embodiments, X is tosylate.

In some embodiments, q is 2.

In some embodiments, the LSD1 inhibitor is

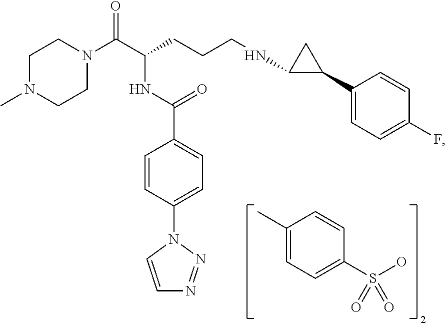

or a polymorph or solvate thereof.

In some embodiments, the LSD1 inhibitor is

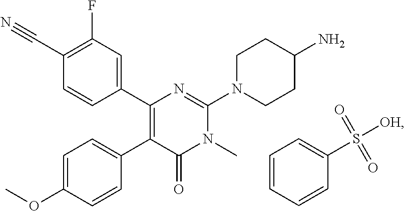

or a polymorph or solvate thereof.

### Bone Marrow Failure and Related Conditions

Provided herein are methods of treatment of a condition characterized by reduced numbers of HSCs such as bone marrow failure in a patient in need thereof comprising: administering an LSD1 inhibitor to said patient. Homeostasis as used herein refers to a state wherein niche factors maintain most hematopoietic stem cells (HSCs) in a quiescent state, which is thought to protect HSCs from malignant transformation and malfunction, as proliferating cells are more susceptible to genetic mutations and will become senescent once their turnovers reach their maximum.

Intrinsic defects include mutations that impair normal HSC function. Extrinsic factors include any pathological alteration in the bone marrow niche that impairs normal functioning of the HSC, and insults to the HSC itself. Extrinsic factors can include autoimmune antibodies or immune cells that target HSCs, the bone marrow itself being altered by a pathologic process, such as leukemia or fibrosis, that renders the bone marrow niche inadequate for HSC survival, exposure of the bone marrow to a toxic insult such as radiation, chemotherapy, infections, idiopathic factors, drugs or other elements, or chemical toxins that kill HSCs, or depletion of HSCs secondary to intrinsic—often genetic—defects that ultimately lead to their premature death.

BMF syndromes impair hematopoiesis, resulting in anemia, thrombocytopenia, and neutropenia, which increases the risk of infection. BMF can also include, e.g., myelodysplastic syndromes, in which immature blood cells in the bone marrow do not mature to become healthy cells, resulting in symptoms of BMF. More than 10,000 people in the US are diagnosed with myelodysplastic syndromes every year, and about a third of those patients will progress to acute myeloid leukemia (AML).

In some embodiments, the specific type of BMF, or the specific BMF syndrome, to be treated as described herein may be a type of BMF that arises from one or more genetic abnormalities described herein, such as in a gene involved in hematopoiesis or one coding for a protein that maintains the integrity of the genome or DNA. A decrease in LSD1 activity may result in increased expression of the genes involved in regulating hematopoiesis, leading to an increase in HSCs, and hence the progeny of the HSCs. In some embodiments, the administration of the LSD1 inhibitor results in the expansion of long-term or short-term bone marrow hematopoietic stem cells (HSCs). In some embodiments, treatment of BMF with an LSD1 inhibitor as described herein results in increased blood cells, such as an increase in red blood cells, or an increase in platelets, or an increase in white blood cells. In some embodiments, administration of an LSD1 inhibitor as described herein results in amelioration or lessening or elimination of symptoms associated with BMF. In some embodiments, administration of an LSD1 inhibitor as described herein results in amelioration or lessening or elimination of symptoms associated with a BMF syndrome.

In some embodiments, a BMF syndrome that can be treated using an LSD1 inhibitor as described herein may be, e.g., Fanconi anemia, dyskeratosis congenita, Shwachman-Diamond syndrome, congenital amegakaryocytic thrombocytopenia, Diamond-Blackfan anemia, or reticular dysgenesis, a GATA2-related disorder, a SAMD9/SAMD9L-related disorder, radiation or chemical poisoning of the bone marrow including chemotherapy, or BMF associated with leukemia, myelofibrosis, sarcoid, granulomatous disease of the bone marrow or myelodysplastic syndrome. In some embodiments, the patient achieves remission for bone failure or a BMF syndrome and the bone failure or a BMF syndrome recurs.

### Hereditary Bone Marrow Failure (BMF)/Myelodysplastic Syndromes (MDS)

BMF can arise from many causes; it can be inherited or acquired after birth. Inherited BMF syndromes are most often a consequence of germline mutations passed down from parents. From a biological perspective, inherited BMF syndromes are caused by loss of function of fundamental cellular pathways such as DNA repair, production and function of ribosomes, or telomere maintenance.

Classical inherited BMF syndromes can be categorized into Diamond-Blackfan anemia (DBA) or Diamond-Blackfan syndrome, Fanconi anemia (FA), severe congenital neutropenia (SCN), dyskeratosis congenita (DC), Shwachman-Diamond syndrome (SDS), congenital thrombocytopenias, and telomere diseases. Most BMF syndromes are inherited in an autosomal recessive manner (e.g., FA, SDS, congenital amegakaryocytic thrombocytopenia, and reticular dysgenesis), while a small subset is inherited in X-linked recessive (dyskeratosis congenita, 2% of FA) or autosomal dominant mode of inheritance (Diamond-Blackfan anemia, reticular dysgenesis).

Myelodysplastic syndromes (MDS) are heterogeneous clonal hematopoietic disorders characterized by ineffective hematopoiesis, bone marrow dysplasia, and peripheral cytopenias. Familial forms of MDS have traditionally been considered rare, especially in adults; however, the increasing availability of somatic and germline genetic analyses has identified multiple susceptibility loci. Within pediatric oncology, there is an understanding of rare inherited predispositions to primary MDS associated with bone marrow failure syndromes such as FA, DC, and SBS in children. To date, there are seven well-defined single-gene loci that, when mutated, predispose to an increased lifetime risk of primary MDS: RUNX1, ANKRD2, DDX41, ETV6, GATA2, SRP72, TERT/TERC, and complementation group.

Development of inherited BMF syndromes can occur via a number of critical points in hematopoietic lineage pathways. Specific mutant alleles known to cause BMF are found in genes that directly affect cell survival and function, which are essential for normal hematopoiesis. Other genetic modifying processes, e.g., cytokine signaling hyperactivity isolated in the pathogenesis of acquired autoimmune aplastic anemia, can also be implicated in BMF. Hematopoietic, gastrointestinal, and integumentary cell lineages are most often affected, as these cells divide frequently.

Some BMF syndromes, e.g., Fanconi anemia, involves genomic instability due to mutations in genes coding for DNA damage repair proteins, which are important for maintenance of genomic integrity, and thus, any alteration or abnormality allows mutations in other genes during the process of normal cell division.

A number of genes can contribute to BMF syndromes, including, but not limited to, a gene in the FANC family, e.g., FANCD1, FANCG, FANCJ, FANCP, which is the most common mutation in Fanconi anemia. Some patients also have mutations in BRCA2, which carries an additional risk associated with breast and ovarian cancer. These proteins are responsible for DNA damage repair via homologous recombination. When these genes are mutated, chromosomal breakage can occur during cell replication, leading to activation of salvage nonhomologous end-joining pathways, bridge-fusion-breakage cycles, and massive aneuploidy. Some proteins also participate in the stem cell survival pathway in a direct or indirect manner, further influencing cell survival.

Mutations in genes encoding proteins and RNAs involved in telomere maintenance (DKC1, TERC, TERT, NOLA2, NOLA3, WRAP53) can result in dyskeratosis congenita. Telomerase is involved in cell replication, so defects in telomerase result in shortened telomeres, leading to premature hematopoietic stem cell exhaustion and marrow aplasia.

Shwachman-Diamond syndrome is caused by mutations in the Shwachman-Bodian-Diamond syndrome (SBDS) gene, which is involved in ribosome biogenesis and mitotic spindle function.

Mutations in the MPL (myeloproliferative leukemia virus) oncogene encoding thrombopoietin receptor c-MPL, which regulates megakaryopoiesis and platelet production, cause thrombocytopenia due to reduced or eliminated megakaryocytes in the bone marrow, leading to congenital amegakaryocytic thrombocytopenia.

Mutations in the RPL and RPS gene families, which encode ribosomal proteins involved in ribosome biogenesis, cause Diamond-Blackfan anemia.

Mutations in the mitochondrial adenylate kinase 2 (AK2) gene cause reticular dysgenesis, which is one of the rarest and most severe forms of severe common immunodeficiency (SCID) in which patients develop profound leukopenia.

### BMF Secondary to Chronic Infiltrative Diseases

BMF can also arise secondary to chronic infiltrative diseases including neoplastic diseases such as myelofibrosis (MF), various forms of leukemia such as AML, sarcoid, and metastatic disease.

### BMF Secondary to Somatic Mutations in HSCs

BMF can also arise in HSCs which acquire mutations that promote increased self-renewal of the stem cell state, increased proliferation, and/or reduced cell death, which have the capacity to expand the HSC clone at a disproportionate rate compared to other clones, a condition termed clonal hematopoiesis (CH). Recognized subtypes of CH include CH of indeterminate potential (CHIP), age-related CH (ARCH), idiopathic cytopenias of undetermined significance (ICUS), and clonal cytopenias of undetermined significance (CCUS).

### Acquired BMF Secondary to Autoimmune Destruction of HSCs

BMF syndromes can also arise as an autoimmune condition in which the HSC becomes the target of activated T-cells that kill HSCs.

### Myelotoxicity Due to Therapeutic or Environmental Insult (Irradiation, Chemotherapy, Toxins)

BMF can also result from chemical or radiation poisoning that kill HSCs such as occurs in the treatment of cancer or exposure to myelotoxic chemicals. Finally, BMF can be a consequence of infiltrative diseases that render bone marrow inhospitable to hematopoiesis such as leukemia.

### BMF Due to Infections

BMF can also arise secondary to infections such as tuberculosis (TB), viral infections (e.g., parvovirus B19, CMV, HSV, etc.), and the like.

The above examples are non-limiting, and other BMF syndromes caused by or associated with other genes or pathways are intended to be encompassed within the scope of the present disclosure.

### Conditions Related to Bone Marrow Failure

Anemia is a condition characterized by a low number of red blood cells, e.g., low hemoglobin or hematocrit. Mild anemia can be classified by a hemoglobin level of 10.0 g/dL to a lower limit of normal hemoglobin, moderate anemia by a hemoglobin level of 8.0-<10.0 g/dL, and severe anemia by a hemoglobin level of 6.5-7.9 g/dL. As described herein, administration of an LSD1 inhibitor provides a treatment for BMF and as a result, prevents conditions such as anemia. In some embodiments, administration of an LSD1 inhibitor to a patient having a BMF syndrome prevents development or exacerbation of anemia by preventing the reduction of red blood cells (i.e., hemoglobin) in the patient, or promoting the production of red blood cells, e.g., by hematopoietic stem cells in the bone marrow. Thus, in some embodiments, administration of an LSD1 inhibitor as described herein does not reduce the population of red blood cells or hemoglobin in the patient, e.g., to mild, moderate, or severe anemia.

In some embodiments, BMF as described herein may be characterized by any symptoms of BMF described herein, e.g., anemia, thrombocytopenia, or any other symptoms of BMF described herein or in the art. In some embodiments, the anemia may be aplastic anemia, in which the bone marrow cells, or bone marrow stem cells, e.g., hematopoietic stem cells, do not produce enough new blood cells, e.g., red blood cells, white blood cells, and/or platelets.

Thrombocytopenia is a condition wherein an individual has a low platelet count. Platelets (i.e., thrombocytes) are blood cells that aid in blood clotting by clumping and forming plugs in injuries involving blood vessels. Thrombocytopenia can generally be classified into grades of severity, with Grade 1 having platelet counts in a range of 75,000-150,000 platelets/mm³, Grade 2 having a range of 50,000-75,000 platelets/mm³, Grade 3 having a range of 25,000-50,000 platelets/mm³, and Grade 4 having less than 25.000 platelets/mm³. Stated differently, Grade 1 thrombocytopenia has a platelet count of 75×10⁹/L-150×10⁹/L, Grade 2 of 50×10⁹/L-75×10⁹/L, Grade 3 of 25×10⁹/L-50×10⁹/L, and Grade 4 having less than 25×10⁹/L. As described herein, administration of an LSD1 inhibitor provides a treatment for BMF and as a result, prevents conditions such as thrombocytopenia. In some embodiments, administration of an LSD1 inhibitor to a patient having BMF or a bone narrow failure syndrome prevents development or exacerbation of thrombocytopenia by preventing the reduction of platelets in the patient, or promoting the production of platelets, e.g., by hematopoietic stem cells in the bone marrow. Thus, in some embodiments, administration of an LSD1 inhibitor as described herein does not reduce the population of platelets in the patient, e.g., to a grade of Grade 1, Grade 2, Grade 3, or Grade 4 thrombocytopenia. In some embodiments, administration of an LSD1 inhibitor as described herein does not reduce the population of platelets in the patient to Grade 4 thrombocytopenia, or to a platelet count of less than 25×10⁹/L.

In a preferred embodiment, the BMF syndrome is any one or a combination of the BMF syndromes chosen from: BMF syndrome secondary to chronic infiltrative diseases including neoplastic diseases, BMF syndrome due to infections, BMF syndrome due to therapeutically-, environmentally-, or irradiation-induced myelotoxicity, heritable BMF syndrome, acquired BMF syndrome secondary to autoimmune destruction, and BMF syndrome secondary to somatic mutations in HSCs. In some embodiments, the BMF syndrome is secondary to an autoimmune condition causing aplastic anemia. In some embodiments, the BMF syndrome is inherited. In some embodiments, the BMF syndrome is Fanconi anemia, dyskeratosis congenita, Shwachman-Diamond syndrome, congenital amegakaryocytic thrombocytopenia, Diamond-Blackfan anemia, reticular dysgenesis, a GATA2-related disorder, or a SAMD9/SAMD9L-related disorder. In some embodiments, the BMF syndrome is Fanconi anemia or Diamond-Blackfan anemia. In some embodiments, the BMF syndrome is caused by near-ablative cancer therapies, for example, chemotherapy or irradiation. In some embodiments, the BMF syndrome is secondary to chronic infiltrative diseases including neoplastic diseases, for example, metastatic cancer. In some embodiments, the BMF syndrome caused by an infection such as tuberculosis or B17 parvovirus. In some embodiments, the BMF syndrome is secondary to somatic mutations in HSCs that cause cytopenias, for example, MDS.

### Lysine-Specific Histone Demethylase 1A (LSD1) Inhibitors for Treatment of BMF Syndromes

Lysine-specific demethylase 1 (or KDM1A) participates in the regulation of HSC proliferation. For HSCs, there is a delicate balance maintained between HSC quiescence and proliferation. In order to minimize HSC entry into the cell cycle for DNA replication and cell division, HSCs are maintained in a state of quiescence to minimize DNA replication errors that arise naturally in the course of replication a genome. HSCs are recruited for proliferation and differentiation on an as-needed basis. This need is enhanced during bone marrow injury or stress.

The inhibition of LSD1 pharmacologically or through genetic knock-down results in the proliferation and expansion of hematopoietic stem and progenitor cells, showing the most pronounced expansion in the LT-HSC compartment. LSD1 knock-down also has been shown to increase the proportion of LT-HSCs actively entering the cell cycle. Contrary to this, LSD1 deletion results in pancytopenia with a reduction of stem and progenitor cells. Remarkably, HSCs do not lose their multipotent potential to differentiate into all blood cell lineages due to inhibition. In short, LSD1 inhibition in an HSC does not induce differentiation. HSCs retain their “stem-like” properties of self-renewal and multipotency.

The administration of an LSD1 enzymatic inhibitor can be used clinically for several purposes. First, an LSD1 inhibitor may be used to increase the number of HSCs that can be harvested from peripheral blood of donors for hematopoietic stem cell transplant (HSCT) in other patients with BMF syndromes, such as leukemia or myelofibrosis. An LSD1 inhibitor could be administered orally or parentally to the donor, i.e., in vivo, after which cells are collected from the peripheral circulation of the donor, or donor HSCs could be exposed to an LSD1 inhibitor and expanded in culture ex vivo. Second, an LSD1 inhibitor may be administered to a patient to increase the number of stem cells in a patient who is deficient in HSCs, for example due bone marrow failure or another condition characterized by reduced numbers of HSCs. Third, an LSD1 inhibitor may be administered to a patient to cause the proliferation of HSCs to enhance recovery from near-ablative cancer therapies and/or allow the usage of higher doses of near-ablative cancer therapies by increasing HSCs during recovery from such treatment.

Useful LSD1 inhibitors for treatment of BMF or a BMF syndrome may be irreversible inhibitors or may be reversible inhibitors with low or high binding affinity for LSD1. In some embodiments, the LSD1 inhibitor is an irreversible inhibitor. In some embodiments, the LSD1 inhibitor is a reversible inhibitor. In some embodiments, the LSD1 inhibitor is an irreversible inhibitor or a reversible inhibitor with low, moderate, or high binding affinity. In some embodiments, the irreversible LSD1 inhibitor is chosen from tranylcypromine (TCP), ORY-1001 (iadademstat), GSK-2879552, IMG-7289 (bomedemstat, Compound 3), INCB059872, CC-90011 (pulrodemstat), T-3775440, TAK-448, TAK-418 and ORI-2001 (vafidemstat). In some embodiments, the reversible LSD1 inhibitor is chosen from Compound 1, Compound 2, Compound 4, CC-90011 and SP-2577. In some embodiments, the LSD1 inhibitor is IMG-7289 (bomedemstat).

In some embodiments, the LSD1 inhibitor is a compound disclosed in WO2016130952, e.g., of the formula (I):

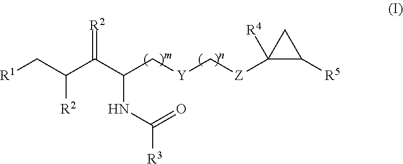

or a salt, polymorph, or solvate thereof, wherein:
   Y is chosen from a bond, NR^{4a}, O, C(O)NH, NHC(O), S, SO₂, CHOH, and CH₂;
   Z is chosen from a bond, NR^{4b}, O, C(O)NH, NHC(O), S, SO₂, and CH₂;
   m is chosen from 0, 1, 2, 3, 4, and 5;
   n is chosen from 0, 1, 2, and 3;
   R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
   R³ is chosen from alkylamino, cycloalkylamino, arylamino, heteroarylamino, heterocycloalkylamino, cycloalkyl, cycloalkylalkyl, aryl, arylalkyl, heteroaryl, heteroarylalkyl, heterocycloalkyl, and heterocycloalkylalkyl any of which may be optionally substituted with between 0 and 3 R⁶ groups;
   R⁴, R⁴, and R^{4b} are independently chosen from hydrogen, alkyl, alkenyl, alkynyl, and cycloalkyl;
   R⁵ is chosen from aryl and heteroaryl, any of which may be optionally substituted with between 0 and 3 R⁶ groups;
   each R⁶ is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
   R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl.

In some embodiments, the compound has Formula V of WO2016130952:

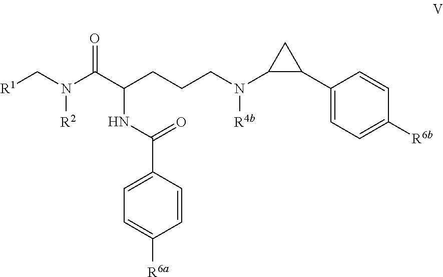

or a salt, polymorph, or solvate thereof, wherein:
   R¹ and R² are each independently chosen from alkyl, aminoalkyl, alkylsulfonylalkyl, alkoxyalkyl, aryl, arylalkyl, cycloalkyl, cycloalkylalkyl, phenyl, biphenyl, heteroaryl, heteroarylalkyl, heterocycloalkyl, and heterocycloalkylalkyl and R¹ and R², together with the nitrogen to which they attach, form a nitrogen-containing heterocycloalkyl or heteroaryl ring, which may be optionally substituted with between 0 and 3 R⁶ groups;
   R^{4b} is chosen from hydrogen, alkyl, alkenyl, alkynyl, and cycloalkyl;
   R^{6a} is chosen from heteroaryl, cyano, and S(O)₂N(CH₃)₂;
   each R⁶ and each R^{6b} is independently chosen from hydrogen, halogen, alkyl, alkylsulfonylaryl, alkenyl, alkynyl, cycloalkyl, haloalkyl, haloalkoxy, haloaryl, alkoxyaryl, aryl, aryloxy, aralkyl, heterocycloalkyl, heteroaryl, alkylheteroaryl, heteroarylalkyl, cyano, alkoxy, alkoxyaryl, amino, alkylamino, dialkylamino, oxo, COR⁷, SO₂R⁷, NHSO₂R⁷, NHSO₂NHR⁷, NHCOR⁷, NHCONHR⁷, CONHR⁷, and CONR⁷R⁸; and
   R⁷ and R⁸ are independently chosen from hydrogen, aryl, and lower alkyl; or R⁷ and R⁸ may be taken together to form a nitrogen-containing heterocycloalkyl or heteroaryl ring, which may be optionally substituted with lower alkyl.

In some embodiments, the LSD1 inhibitor is chosen from amongst the compound Examples disclosed in WO2016130952, or a salt, polymorph, or solvate thereof.

In some embodiments, the LSD1 inhibitor is

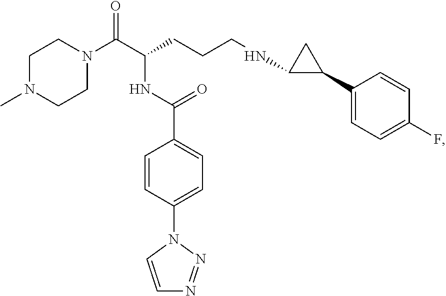 [SMILES: CN1CCN(C(=O)[C@H](CCCN[C@@H]2C[C@H]2c2ccc(F)cc2)NC(=O)c2ccc(-n3ccnn3)cc2)CC1]

or a salt, polymorph, or solvate thereof.

In some embodiments, the LSD1 inhibitor is a salt of the formula:

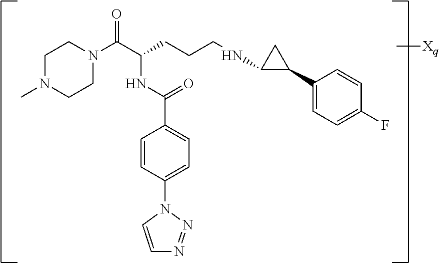

or a polymorph or solvate thereof, wherein:
   X is chosen from tosylate, sulfate, tartrate, oxalate, besylate, fumarate, citric, esylate, and malate; and
q is an integer chosen from 1 and 2.

In some embodiments, X is tosylate.

In some embodiments, q is 2.

In some embodiments, the LSD1 inhibitor is

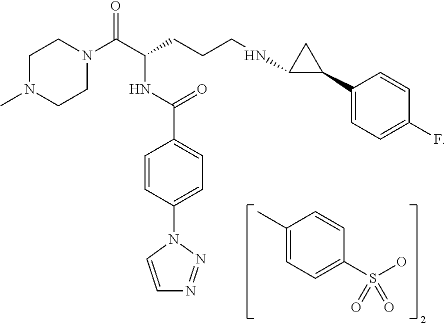

(i.e., bomedemstat, IMG-7289, Compound 3) or a polymorph or solvate thereof.

In some embodiments, the LSD1 inhibitor is a compound of structural Formula (Ia):

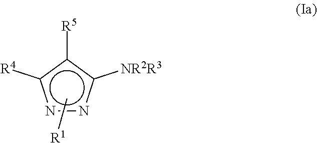

or a salt or tautomer thereof, wherein:
   R¹ is chosen from cycloalkyl, heterocycloalkyl, aryl, and heteroaryl, any of which is optionally substituted with one R⁶, and one or more R⁷;
   R² and R³ are independently chosen from H, cycloalkyl, heterocycloalkyl, aryl, heteroaryl, (cycloalkyl)alkyl. (heterocycloalkyl)alkyl, (aryl)alkyl, and (heteroaryl)alkyl, any of which is optionally substituted with one or more R⁸, or R² and R³, together with the intervening nitrogen, combine to form heterocycloalkyl which is optionally substituted with one or more R⁸, —R⁴ is chosen from cycloalkyl, heterocycloalkyl, aryl, and heteroaryl, any of which is optionally substituted with one or more R⁹;
   R⁵ is chosen from H, halo, and cyano;
   R⁶ is chosen from cycloalkyl, heterocycloalkyl, aryl, heteroaryl. (cycloalkyl)carbonyl, (heterocycloalkyl)carbonyl, (aryl)carbonyl, and (heteroaryl)carbonyl, any of which is optionally substituted with one or more R¹⁰;
   each R⁷ is independently chosen from halo, cyano, hydroxy, alkyl, and alkoxy;
   each R⁸ is independently chosen from —NHR¹¹, —C(NH)NHR¹¹, —NHC(═NH)—R¹¹, —NHC(═NH)NHR¹¹, —CONHR¹¹, —CH₂NHR¹¹, —CH₂C(═NH)NHR¹¹, —CH₂NHC(═NH)—R¹¹, —CH₂NHC(═NH)NHR¹¹, —CH₂CONHR¹¹, halo, cyano, hydroxy, alkyl, and alkoxy;
   each R⁹ is independently chosen from halo, cyano, hydroxy, alkyl, and alkoxy; and
   each R¹⁰ is independently chosen from halo, cyano, hydroxy, oxo, alkyl, alkoxy, (hydroxy)alkyl. (hydroxy)alkoxy, alkylthio, and alkylsulfonyl; and
   each R¹¹ is independently chosen from H and alkyl.

In some embodiments, the LSD1 inhibitor is a compound of structural Formula (IIa):

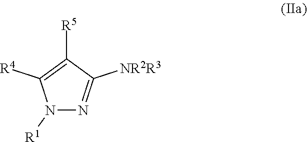

or a salt or tautomer thereof, wherein:
   R¹ is chosen from cycloalkyl, heterocycloalkyl, aryl, and heteroaryl, any of which is optionally substituted with one R⁶, and one or more R⁷;
   R² and R³ are independently chosen from H, cycloalkyl, heterocycloalkyl, aryl, heteroaryl, (cycloalkyl)alkyl. (heterocycloalkyl)alkyl, (aryl)alkyl, and (heteroaryl)alkyl, any of which is optionally substituted with one or more R⁸,
   or R² and R³, together with the intervening nitrogen, combine to form heterocycloalkyl which is optionally substituted with one or more R⁸;
   R⁴ is chosen from cycloalkyl, heterocycloalkyl, aryl, and heteroaryl, any of which is optionally substituted with one or more R⁹;
   R⁵ is chosen from H, halo, and cyano;
   R⁶ is chosen from cycloalkyl, heterocycloalkyl, aryl, heteroaryl. (cycloalkyl)carbonyl, (heterocycloalkyl)carbonyl, (aryl)carbonyl, and (heteroaryl)carbonyl, any of which is optionally substituted with one or more R¹⁰;
   each R⁷ is independently chosen from halo, cyano, hydroxy, alkyl, and alkoxy;
   each R⁸ is independently chosen from —NHR¹¹, —C(NH)NHR¹¹, —NHC(═NH)—R¹¹, —NHC(═NH)NHR¹¹, —CONHR¹¹, —CH₂NHR¹¹, —CH₂C(═NH)NHR¹¹, —CH₂NHC(═NH)—R¹¹, —CH₂NHC(═NH)NHR¹¹, —CH₂CONHR¹¹, halo, cyano, hydroxy, alkyl, and alkoxy;
   each R⁹ is independently chosen from halo, cyano, hydroxy, alkyl, and alkoxy; and
   each R¹⁰ is independently chosen from halo, cyano, hydroxy, oxo, alkyl, alkoxy, (hydroxy)alkyl. (hydroxy)alkoxy, alkylthio, and alkylsulfonyl; and
   each R¹¹ is independently chosen from H and alkyl.

In some embodiments, the LSD1 inhibitor is chosen from amongst the compound Examples disclosed in WO2016130952, or a salt, polymorph, or solvate thereof.

In some embodiments, the LSD1 inhibitor is

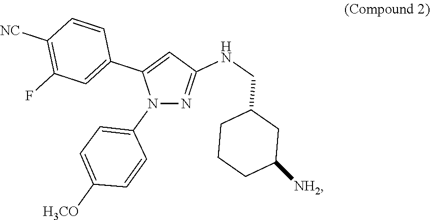 [SMILES: COc1ccc(-n2nc(NC[C@H]3CCC[C@H](N)C3)cc2-c2ccc(C#N)c(F)c2)cc1]

or a salt, polymorph, or solvate thereof.

In some embodiments, the LSD1 inhibitor is

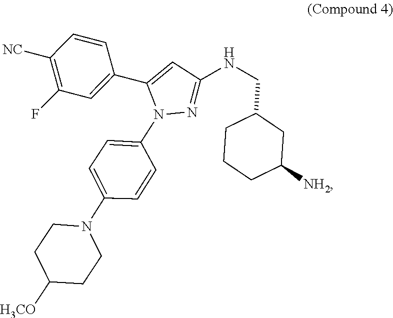

or a salt, polymorph, or solvate thereof.

In some embodiments, the LSD1 inhibitor is a compound disclosed in WO2015168466, e.g., of the formula (Ib):

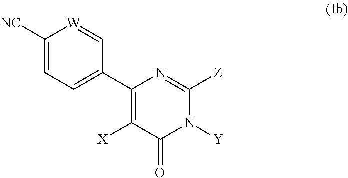

or a salt, polymorph, solvate, or tautomer thereof, wherein,
   W is N, C—H, or C—F;
   X is hydrogen, halogen, —CN, optionally substituted alkyl, optionally substituted alkynyl, optionally substituted carbocyclylalkynyl, optionally substituted aryl, or optionally substituted heteroaryl;
   Y is hydrogen, optionally substituted alkyl, optionally substituted cycloalkyl, or optionally substituted cycloalkylalkyl;
   Z is an optionally substituted group chosen from alkyl, carbocyclyl, C-attached heterocyclyl, N-attached heterocyclyl, heterocyclylalkyl, heterocyclylalkenyl, —O-heterocyclyl,
      —N(R)-heterocyclyl, —O-heterocyclylalkyl, —N(R)-heterocyclylalkyl, —N(R)(C₁-C₄alkylene)-NR₂.
      —O(C₁-C₄alkylene)-NR₂; and
   R is hydrogen or C₁-C₄alkyl.

In some embodiments, the LSD1 inhibitor is

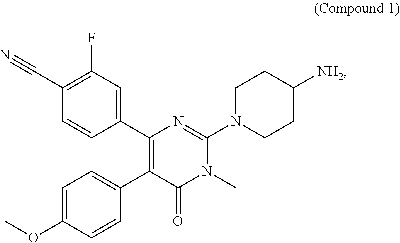 [SMILES: COc1ccc(-c2c(-c3ccc(C#N)c(F)c3)nc(N3CCC(N)CC3)n(C)c2=O)cc1]

or a salt or tautomer thereof.

In some embodiments, the Compound 1 is the besylate salt of Compound 1:

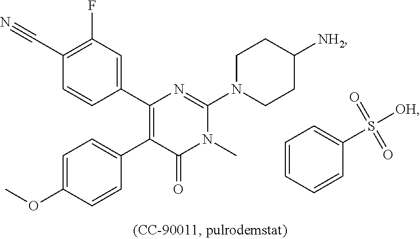

or a polymorph or solvate thereof.

Additional LSD1 inhibitors are known in the art, and include, e.g., those disclosed in the following references:

| | | |
|---|---|---|
| WO2014/164867A1 | WO2012/013728A1 | US2012/0322877A1 |
| WO2015/021128A1 | WO2012/013727A1 | US2015/0225394A1 |
| WO2016/130952A1 | WO2012/042042A1 | US2015/0225401A1 |
| U.S. Pat. No. 9,388,123 | WO2012/045883A1 | US2015/0225375A1 |
| WO2010/143582A1 | WO2012/072713A2 | US2015/0225379A1 |
| WO2012/135113A2 | WO2012/107499A1 | US2016/0009711A1 |
| WO2009/027349A2 | WO2012/107498A1 | US2016/0009712A1 |
| WO2010/043721A1 | WO2012/156537A2 | US2016/0009720A1 |
| WO2010/084160A1 | WO2012/156531A2 | US2016/0009721A1 |
| WO2011/035941A1 | WO2013/057320A1 | WO2016/161282A1 |
| WO2011/042217A1 | WO2013/057322A1 | US2015/0315126A1 |
| WO2011/106105A2 | U.S. Pat. No. 8,765,820 | US2015/0065495A1 |
| WO2011/106106A2 | U.S. Pat. No. 8,389,580 | WO2015/120281A1 |
| WO2011/106573A2 | US2016/0120862A1 | WO2016/123387A1 |
| WO2011/106574A2 | US2015/0025054A1 | WO2016172496A1 |
| WO2011/131697A1 | WO2015/134973A1 | US2015/0065495A1 |
| WO2012/013728A1 | US2014/0011857A1 | |

the contents of which are hereby incorporated by reference as if written herein in their entireties.

### Methods of Treatment of BMF and Related Conditions

### Pharmaceutical Compositions

In some embodiments, a composition comprising an LSD1 inhibitor is provided. In some embodiments, an LSD1 inhibitor may be administered as a single composition. In some embodiments, each drug may be administered separately (while still being administered concurrently), i.e., in separate solutions or drug forms as described herein. For example, a LSD1 inhibitor and/or a second drug or drug regimen as described herein may be administered to a patient in an aqueous solution for intravenous administration, or they may be formulated for oral administration. In other embodiments, the LSD1 inhibitor and the second drug regimen may be administered in separate or distinct aqueous solutions for intravenous administration. Pharmaceutical formulation is well established and known in the art.

In some embodiments, an LSD1 inhibitor and/or a second drug or drug regimen may be formulated with excipient materials, such as sodium citrate, sodium dibasic phosphate heptahydrate, sodium monobasic phosphate, Tween-80 (polyethylene glycol sorbitan monooleate, CAS Number 9005-65-6), and/or a stabilizer. The LSD1 inhibitor and/or second drug regimen can be provided, for example, in a buffered solution at a suitable concentration and can be stored at an appropriate temperature to maintain the efficacy of the drug(s), for example a temperature of 2-8° C. In some other embodiments, the pH of the composition is between about 5.5 and about 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5).

A pharmaceutical composition can also include agents that reduce aggregation of the drug when formulated. Examples of aggregation reducing agents include one or more amino acids chosen from methionine, arginine, lysine, aspartic acid, glycine, and glutamic acid. The pharmaceutical compositions can also include a sugar (e.g., sucrose, trehalose, mannitol, sorbitol, or xylitol) and/or a tonicity modifier (e.g., sodium chloride, mannitol, or sorbitol) and/or a surfactant (e.g., polysorbate-20 or polysorbate-80).

As described above for LSD1 inhibitors, compositions comprising the LSD1 inhibitor and/or the second drug or drug regimen can be administered by a parenteral mode (e.g., intravenous, subcutaneous, intraperitoneal, or intramuscular injection). In one embodiment, a composition comprising a LSD1 inhibitor is administered orally or intravenously. In other embodiments, the LSD1 inhibitor is administered orally, and the second drug or drug regimen is administered intravenously. The phrases “parenteral administration” and “administered parenterally” as used herein mean modes of administration, usually by injection, and include, without limitation, intravenous, intramuscular, intra-arterial, intrathecal, intracapsular, intraocular, intracardiac, intradermal, intraperitoneal, transtracheal, subcutaneous, subcuticular, intraarticular, subcapsular, subarachnoid, intraspinal, epidural, and intrasternal injection, and infusion.

A composition comprising a LSD1 inhibitor and/or a second drug or drug regimen can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In some embodiments, compositions comprising an LSD1 inhibitor and/or a second drug or drug regimen may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In some embodiments, a composition comprising a LSD1 inhibitor and/or a second drug or drug regimen is formulated in sterile distilled water or phosphate buffered saline. The pH of the pharmaceutical formulation may be between about 5.5 and about 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5).

### Administration of LSD1 Inhibitors

An LSD1 inhibitor as described herein can be administered to a subject, e.g., a patient having a condition characterized by reduced numbers of HSCs such as BMF or a BMF syndrome, by a variety of methods. For many applications, the route of administration is one of: oral administration, intravenous injection or infusion (IV), subcutaneous injection (SC), intraperitoneally (IP), or intramuscular injection. In some embodiments, an intravenous infusion may be administered on a continuous basis (i.e., a continuous infusion), or may be administered in multiple individual infusions. Other modes of parenteral administration can also be used. Examples of such modes include: intra-arterial, intrathecal, intracapsular, intraocular, intracardiac, intradermal, transtracheal, subcuticular, intra-articular, subcapsular, subarachnoid, intraspinal, and epidural and intrasternal injection.

The route and/or mode of administration of the LSD1 inhibitor, or compositions comprising these, can also be tailored for the individual case, e.g., by monitoring the patient.

As would be understood by one of skill in the art, an LSD1 inhibitor as described herein is administered in any form necessary or useful to the subject for treatment of BMF or a BMF syndrome, for example, a liquid (e.g., injectable and infusible solutions), a semi-solid, a solid, an aqueous solution, a suspension, an emulsion, a gel, a magma, a mixture, a tincture, a powder, a capsule, a dispersion, a tablet, a pellet, a pill, a powder, a liposome, a lozenge, a troche, a liniment, an ointment, a lotion, a paste, a suppository, a spray, an inhalant, or the like. In some embodiments, a drug as described herein for treatment of BMF or a BMF syndrome may be administered in a liquid or aqueous form for injection into a patient, or in a pill or tablet form for oral administration. The dosage form of a drug described herein can depend on the intended mode of administration and therapeutic application. Typically, dosage forms for the drug treatments described herein are in the form of injectable or infusible solutions, or in the form of a pill for oral administration.

The composition(s) comprising an LSD1 inhibitor can be administered as a fixed dose, or in a patient-tailored dose, such as on the basis of body weight or mass or based on disease severity. For example, an LSD1 inhibitor may be administered to a patient on a mg/kg basis. The dose can also be chosen to reduce or avoid resistance to the LSD1 inhibitor, or to reduce or avoid production of antibodies against the LSD1 inhibitor. Dosages are adjusted to provide the desired response. e.g., a therapeutic response or a combinatorial therapeutic effect. Generally, doses of the LSD1 inhibitor and/or the second drug or drug regimen can be used in order to provide a subject with the agent in bioavailable quantities.

In some embodiments, a dose of an LSD1 inhibitor may be any dose deemed appropriate by a clinician or practitioner, such as including, but not limited to, a dose of about 0.1 mg/kg to about 2 mg/kg, including, e.g., 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 1.25 mg/kg, about 1.5 mg/kg, about 1.75 mg/kg, about 2 mg/kg. In some embodiments, a dose of an LSD1 inhibitor may be about 2 mg/kg to about 7 mg/kg, such as about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, or about 7 mg/kg. In other embodiments, a dose of an LSD1 inhibitor may include, but is not limited to, a dose of about 3 mg/kg to about 6 mg/kg, such as about 3 mg/kg, about 3.5 mg/kg, about 4 mg/kg, about 4.5 mg/kg, about 5 mg/kg, about 5.5 mg/kg, or about 6 mg/kg. In some embodiments, an LSD1 inhibitor may be administered at a total daily dose of between 20 and 120 mg/day, such as including, but not limited to, a total daily dose of 20 mg, or 21 mg, or 22 mg, or 23 mg, or 24 mg, or 25 mg, or 26 mg, or 27 mg, or 28 mg, or 29 mg, or 30 mg, or 31 mg, or 32 mg, or 33 mg, or 34 mg, or 35 mg, or 36 mg, or 37 mg, or 38 mg, or 39 mg, or 40 mg, or 41 mg, or 42 mg, or 43 mg, or 44 mg, or 45 mg, or 46 mg, or 47 mg, or 48 mg, or 49 mg, or 50 mg, or 51 mg, or 52 mg, or 53 mg, or 54 mg, or 55 mg, or 56 mg, or 57 mg, or 58 mg, or 59 mg, or 60 mg, or 61 mg, or 62 mg, or 63 mg, or 64 mg, or 65 mg, or 66 mg, or 67 mg, or 68 mg, or 69 mg, or 70 mg, or 71 mg, or 72 mg, or 73 mg, or 74 mg, or 75 mg, or 76 mg, or 77 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, or 31 mg, In some embodiments, a dose of an LSD1 inhibitor as described herein, when administered to a patient having BMF or a BMF syndrome, does not result in hematological deficiencies in the patient, such as significantly reducing the population of platelets in the patient.

An LSD1 inhibitor as described herein can be administered, e.g., at a periodic interval over a period of time (a course of treatment) sufficient to encompass at least 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 does, 20 doses, or more, e.g., once daily, twice daily, three times daily, or about one to four times per week, or such as weekly, biweekly (every two weeks), every three weeks, monthly, e.g., for between about 1 to 12 weeks, such as between 2 to 8 weeks, such as between about 3 to 7 weeks, such as for about 4, 5, or 6 weeks, or every 5 weeks, or every 6 weeks, or any interval deemed appropriate by a clinician. Factors that may influence the dosage and timing required to effectively treat a subject, include, e.g., the stage or severity of the disease or disorder, formulation, route of delivery, previous treatments, the general health and/or age of the subject, and other diseases present. Moreover, treatment of a subject with a therapeutically effective amount of an LSD1 inhibitor, or compositions comprising these, can include a single treatment or can include a series of treatments. Similar consideration may be taken when administering a second drug regimen for treating BMF or a BMF syndrome, such as a drug described herein or known or available in the art, regardless of the mode of administration.

In some embodiments, an LSD1 inhibitor as described herein may be administered to a patient for a period of time as described herein, such as for one week, or for a specified number of days. In some embodiments, LSD1 inhibition may then be continued along with, or be followed by, or follow, the administration of a second therapeutic regimen such as a drug, drug combination, or course of irradiation.

In some embodiments, an LSD1 inhibitor may be administered to a patient having BMF or a BMF syndrome for a period of time. In some embodiments, and to the extent deemed necessary or appropriate by a clinical or medical professional, an LSD1 inhibitor may be administered to a patient for a period of time, after which point a second therapy, e.g., a drug, drug combination, or course of irradiation, for treating BMF may be administered to the patient. In some embodiments, the period of time for administration of the LSD1 inhibitor is one cycle. In some embodiments, one cycle is from about 1 day to about 7 days. In some embodiments, a cycle is about 5 days. In some embodiments, the LSD1 inhibitor may be administered to the patient for a period of time from about 1 to about 7 days, as deemed appropriate by a clinician or physician. For example, in some embodiments, a period of time (e.g., one cycle) for administration of an LSD1 inhibitor may include, but is not limited to, 1 day, 2 days, 3 days, 4 days, 5 days, 6 days, 7 days, 8 days, 9 days, 10 days, or the like. In some embodiments, the LSD1 inhibitor is administered to the patient for 5 days. The specific length of time with which a patient having BMF or a BMF syndrome is treated with an LSD1 inhibitor and/or a second drug regimen is determined by the clinician or practitioner.

In some embodiments, the period of time (e.g., cycle) for administration of an LSD1 inhibitor may be repeated after the period of time for an additional 1 to 10 cycles, including 1 cycle, 2 cycles, 3 cycles, 4 cycles, 5 cycles, 6 cycles, 7 cycles, 8 cycles, 9 cycles, 10 cycles, or the like. Thus, as described herein, an LSD1 inhibitor may initially be administered for anywhere from 1 to 10 days, or 1 to 9 days, or 1 to 8 days, or 1 to 7 days, or 1 to 6 days, or 1 to 5 days, or 1 to 4 days, or 1 to 3 days, or 1 to 2 days, or the like, followed by an additional 1 to 10 cycles, with each of the additional 1 to 10 cycles consisting of from 1 to 7 days, or with each of the additional 1 to 10 cycles consisting of about 5 days.

In some embodiments, administration of the LSD1 inhibitor is terminated after the period of time. In some embodiments, administration of the LSD1 inhibitor is continued after the period of time. In some embodiments, the interval between administration of an LSD1 inhibitor and a second treatment for BMF or a BMF syndrome, e.g., administration of a second therapy, e.g., a drug or drug combination, or irradiation, described herein, may be one to three cell cycle divisions. In some embodiments, such a time period may be 24-96 hours, or more. For example, an interval between administration of an LSD1 inhibitor and a second drug or combination of drugs may be 6 hours, or 12 hours, or 18 hours, or 24 hours, or 30 hours, or 36 hours, or 42 hours, or 48 hours, or 54 hours, or 60 hours, or 66 hours, or 72 hours, or 78 hours, or 84 hours, or 90 hours, or 96 hours, or 102 hours, 108 hours, or 114 hours, 120 hours. In some embodiments, the interval may be 1 day, or 2 days, or 3 days, or 4 days, or 5 days, or 6 days, or 7 days, or 8 days, or 9 days, or 10 days, or 11 days, or 12 days, or 13 days, or 14 days, or longer. An appropriate time period or interval between administration of an LSD1 inhibitor and a second drug treatment may be any length of time deemed appropriate by a clinician or practitioner.

### Combination Therapy

An LSD1 inhibitor can be administered to a patient in need thereof, e.g., a patient that has had or is at risk of having a condition characterized by reduced numbers of HSCs such as BMF or a BMF syndrome, alone or in combination with (i.e., by co-administration or sequential administration) a second drug or regimen, or other therapeutic treatments, or procedures for treating a condition characterized by reduced numbers of HSCs such as BMF or a BMF syndrome. In some embodiments, the additional therapeutic treatments or drugs are included in a pharmaceutical composition as described herein. In other embodiments, the additional therapeutic treatments or drugs are co-administered, administered concurrently, or administered sequentially in separate or distinct compositions. In some embodiments, an LSD1 inhibitor and second therapeutic drug, regimen, therapy, or procedure as described herein may be administered in a clinical setting or may be administered in an alternate setting as deemed appropriate by a clinician or practitioner.

In some embodiments, a drug or regimen as described herein for treatment of a condition characterized by reduced numbers of HSCs such as BMF or a BMF syndrome in a patient may be administered by any route or mode of administration, such as intraperitoneal, intravenous, oral, sublingual, rectal, vaginal, ocular, otic, nasal, cutaneous, enteral, epidural, intra-arterial, intravascular, nasal, respiratory, subcutaneous, topical, transdermal, intramuscular, or the like. In some embodiments, an LSD1 inhibitor as described herein is administered orally (PO). In other embodiments, a second drug or drug regimen described or disclosed herein may be in the form of an aqueous solution for intravenous administration.

In some embodiments, the second drug, regimen, therapy, or procedure as described herein may be any standard-of-care drug or drug regimen known or available in the art for treatment of a condition characterized by reduced numbers of HSCs such as BMF or a BMF syndrome, including, but not limited to, supportive care, blood transfusion, stem cell transplantation or bone marrow transplantation, immunosuppressants, bone marrow stimulants, antibiotics, antivirals, androgens, or the like.

In some embodiments, supportive care, including transfusions of red cells or platelets, may be performed in combination with administration of an LSD1 inhibitor as described herein, to reduce signs and symptoms of disease; red blood cells raise blood cell counts and help relieve anemia or fatigue, and platelets help prevent excess bleeding. In some embodiments, blood transfusion may be combined with other drugs to control iron levels as needed. In some embodiments, blood transfusion may also be combined with immunosuppressants to prevent development of antibodies to transfused blood cells. In some embodiments, blood transfusion may be combined with both iron-controlling drugs and immunosuppressants as needed or as deemed appropriate by a medical professional. Growth factors that stimulate the proliferation of specific lineages, such as erythropoietin or androgens for enhancing red cell production, and thrombopoietics for platelet production, granulocyte colony stimulating factors for neutrophil production can be administered.

In some embodiments, hematopoietic stem cell transplantation (HSCT) or bone marrow transplantation may be performed in combination with administration of an LSD1 inhibitor as described herein in order to replace hematopoietic cells in a patient. HSCT involves the infusion of healthy blood stem cells derived from a donor into the patient to re-populate the bone marrow with healthy stem cells and restore the production of blood cells. In allogeneic HSCT, cells are collected from a tissue-matched donor, e.g., often a sibling, or an unrelated donor, whose human leukocyte antigens (HLA) are a matched. Such a match is essential to minimize the donor-derived white blood cells from attacking host cells. In cases of leukemia or other proliferative or infiltrative disorders of bone marrow, chemotherapy and/or radiation is often administered prior to transplantation to kill diseased blood stem cells or the cells mediating the destruction of healthy stem cells. Ablation of the blood cells in the marrow helps recreate the bone marrow niche enhancing the probability that the transplanted cells will engraft and support new blood cell production. Agents such as cyclosporin are then administered to suppress the immune system to prevent rejection of the donor cells if residual immune cells remain in the host or if the donor cells result in an immune response to the host cells. Stem cell or bone marrow transplantation may be preferable for patients under certain conditions, such as age, health status, availability of an appropriate donor, or the like. In some embodiments, transplantation may be combined with depletion of the patient's failing bone marrow using, e.g., radiation treatment or chemotherapy.

In some embodiments, a transplantation patient may be administered drugs to prevent rejection of the transplanted cells, e.g., an immunosuppressant described herein in combination with an LSD1 inhibitor. Immunosuppressants are known in the art for this purpose and may be used as deemed appropriate by a medical professional. Exemplary immunosuppressants may include, but are not limited to, drugs such as cyclosporine (Gengraf, Neoral, Sandimmune) and anti-thymocyte globulin. In some embodiments, administration of an immunosuppressant may help the bone marrow recover and generate new hematopoietic cells. In some embodiments, more than one immunosuppressant may be administered. In some embodiments, cyclosporine and anti-thymocyte globulin may be used. In some embodiments, an immunosuppressant may be combined with a corticosteroid, such as methylprednisolone (Medrol®, Solu-Medrol®).

Additionally, patients undergoing HSCT can be severely immunocompromised and require intense supportive treatment, especially in the several weeks or months following transplant. In some embodiments, bone marrow stimulants may be administered in combination with administration of an LD1 inhibitor as described herein for treatment of BMF or a BMF syndrome as described herein. Bone marrow stimulants are known in the art for this purpose and may be used as deemed appropriate by a medical professional. Exemplary bone marrow stimulants may include, but are not limited to, colony-stimulating factors, such as sargramostim (Leukine®), filgrastim (Neupogen®) and pegfilgrastim (Neulasta®), epoetin alfa (Epogen®/Procrit®), eltrombopag (Promacta®), or the like. In some embodiments, bone marrow stimulants help stimulate the bone marrow to produce new blood cells. In some embodiments, growth factors are administered with immunosuppressants. Also, in the case of autoimmune destruction of HSCs, immunosuppression may be used for patients who are not eligible for stem cell transplantation or for whom a suitable donor is not available.

In some embodiments, antibiotics or antivirals may be administered in combination with administration of an LD1 inhibitor. Antibiotics and antivirals are known in the art may be used as deemed appropriate by a medical professional.

In some embodiments, androgens may be administered in combination with administration of an LD1 inhibitor. Any androgens known or available in the art may be used as deemed appropriate by a medical professional, for example including, but not limited to, danazol (Cyclomen®), oxandrolone (Oxandrin®), oxymetholone (Anadrol®), fluoxymesterone (Halotestin®), and nandrolone decanoate. In some embodiments, an androgen may be a non-masculinizing androgen, such as Danazol.

In some embodiments, an LSD1 inhibitor as described herein can be administered concurrently with a second drug regimen, such as one or more of granulocyte colony-stimulations factor (G-CSF), anti-thymocyte globulin, eltrombopag (Promacta®), and/or cyclosporin. In some embodiments, for patients developing cancer from advancing BMF, e.g., caused by a myelodysplastic syndrome, an LSD1 inhibitor as described herein can be administered concurrently with a chemotherapeutic regimen known and available in the art such as azacytidine. In other embodiments, the LSD1 inhibitor can be administered for a period of time as described herein, and then discontinued before administration of the second drug regimen. In some embodiments, the LSD1 inhibitor may be administered during the recovery period. In some embodiments, such administration may enhance regeneration of cells in the bone marrow.

In some embodiments, any drug treatments appropriate for treatment of BMF or a BMF syndrome may be used in combination with an LSD1 inhibitor as described herein, and as deemed appropriate by a clinician. For patients who develop cancer as a result of having BMF or a BMF syndrome, a chemotherapeutic drug or drug regimen may be administered. Drugs known in the art for treatment of cancer include, but are not limited to, Evista (Raloxifene Hydrochloride), Raloxifene Hydrochloride, Soltamox (Tamoxifen Citrate), Tamoxifen Citrate, Abemaciclib, Abraxane (Paclitaxel Albumin-stabilized Nanoparticle Formulation), Ado-Trastuzumab Emtansine, Afinitor (Everolimus), Afinitor Disperz (Everolimus), Alkeran (Melphan), Alpelisib, Anastrozole, Aredia (Pamidronate Disodium), Arimidex (Anastrozole), Aromasin (Exemestane), Atezolizumab, Avastin (Bevacizumab), Bevacizumab, Capecitabine, Carboplatin, Cisplatin, Cyclophosphamide, Docetaxel, Doxorubicin Hydrochloride, Doxil (Doxorubicin Hydrochloride Liposome), Ellence (Epirubicin Hydrochloride), Enhertu (Fam-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Femara, (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Hycamtin (Topotecan Hydrochloride), Ibrance (Palbociclib), Infugem (Gemcitabine Hydrochloride), Ixabepilone, Ixempra (Ixabepilone), Kadcyla (Ado-Trastuzumab Emtansine), Keytruda (Pembrolizumab), Kisqali (Ribociclib), Lapatinib Ditosylate, Letrozole, Lynparza (Olaparib), Margenza (Margetuximab-cmkb), Margetuximab-cmkb, Megestrol Acetate, Melphalan, Methotrexate Sodium, Neratinib Maleate, Nerlynx (Neratinib Maleate), Niraparib Tosylate Monohydrate, Olaparib, Paclitaxel, Paclitaxel Albumin-stabilized Nanoparticle Formulation, Palbociclib, Pamidronate Disodium, Pembrolizumab, Perjeta (Pertuzumab), Pertuzumab, Pertuzumab, Rubraca (Rucaparib Camsylate), Trastuzumab, and Hyaluronidase-zzxf, Phesgo (Pertuzumab, Trastuzumab, and Hyaluronidase-zzxf), Piqray (Alpelisib), Ribociclib, Sacituzumab Govitecan-hziy, Soltamox (Tamoxifen Citrate), Talazoparib Tosylate, Talzenna (Talazoparib Tosylate), Tamoxifen Citrate, Taxol, Taxotere (Docetaxel), Tecentriq (Atezolizumab), Tepadina (Thiotepa), Thiotepa, Topotecan Hydrochloride, Toremifene, Trastuzumab, Trastuzumab and Hyaluronidase-oysk, Trexall (Methotrexate Sodium), Trodelvy (Sacituzumab Govitecan-hziy), Tucatinib, Tukysa (Tucatinib), Tykerb (Lapatinib Ditosylate), Verzenio (Abemaciclib), Vinblastine Sulfate, Xeloda (Capecitabine), Zejula (Niraparib Tosylate Monohydrate), Zoladex (Goserelin Acetate). Any other drugs known or available in the art may also be used in combination with an LSD1 inhibitor as described herein without deviating from the scope of the present disclosure.

### Kits

An LSD1 inhibitor for treatment of a condition characterized by reduced numbers of HSCs such as BMF or a BMF syndrome in a patient can be provided in a kit. In one embodiment, the kit includes (a) a container that contains the LSD1 inhibitor as described herein, and optionally (b) informational material. The informational material can be descriptive, instructional, marketing, or other material that relates to the methods described herein and/or the use of the agents for therapeutic benefit.

In some embodiments, the kit also includes a second agent (e.g., a second drug described herein) for treating a condition characterized by reduced numbers of HSCs such as BMF or a BMF syndrome described herein. For example, the kit includes a first container that contains the LSD1 inhibitor, and a second container that includes the second drug or drug regimen. In another embodiment, the kit includes a first container that contains the LSD1 inhibitor, and a second container that contains the second drug or drug regimen.

The informational material of the kits is not limited in its form. In one embodiment, the informational material can include information about production of the compound, molecular weight of the compound, concentration, date of expiration, batch, or production site information, and so forth. In one embodiment, the informational material relates to methods of administering the LSD1 inhibitor, as well as the second drug described herein, e.g., in a suitable dose, dosage form, or mode of administration (e.g., a dose, dosage form, or mode of administration described herein), to treat a subject who has BMF or a BMF syndrome. The information can be provided in a variety of formats, include printed text, computer readable material, video recording, or audio recording, or information that provides a link or address to substantive material, e.g., on the internet.

In addition to the LSD1 inhibitor, and including the second drug if applicable, the kit can include other ingredients, such as a solvent or buffer, a stabilizer, or a preservative. The LSD1 inhibitor, and second drug, can be provided in any form described herein, e.g., liquid, dried or lyophilized form, substantially pure and/or sterile. In some embodiments, when the agents are provided in a liquid solution, the liquid solution is an aqueous solution. When the agents are provided as a lyophilized product, the lyophilized powder is generally reconstituted by the addition of a suitable solvent. The solvent, e.g., sterile water or buffer (e.g., PBS), can optionally be provided in the kit.

The kit can include one or more containers for the drugs or compositions. In some embodiments, the kit contains separate containers, dividers, or compartments for the drugs and informational material. For example, the LSD1 inhibitor, and second drug, if applicable, can be contained in a bottle, vial, or syringe, and the informational material can be contained in a plastic sleeve or packet. In other embodiments, the separate elements of the kit are contained within a single, undivided container. For example, the LSD1 inhibitor, and second drug, if applicable, is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the LSD1 inhibitor, and second drug, if applicable. e.g., in a desired ratio. For example, the kit may include a plurality of syringes, ampules, foil packets, blister packs, or medical devices, e.g., each containing a single combination unit dose. The containers of the kits can be air-tight, waterproof (e.g., impermeable to changes in moisture or evaporation), and/or light-tight.

The kit optionally includes a device suitable for administration of the LSD1 inhibitor, and second drug, if applicable, e.g., a syringe or other suitable delivery device. The device can be provided pre-loaded with one or both of the agents or can be empty, but suitable for loading.

### Definitions

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Any discrepancy between the disclosure of a reference discussed herein and the present disclosure shall be resolved in favor of the present disclosure. Further, the dates of publication provided may be different from the actual publication dates which may need to be independently confirmed.

As used in this specification and the appended claims, the singular forms “a,” “an,” and “the,” along with similar references used in the context of describing a particular embodiment (especially in the context of certain of the following claims), can be construed to cover both the singular and the plural, unless specifically noted otherwise. Thus, for example. “an active agent” refers not only to a single active agent, but also to a combination of two or more different active agents, “a dosage form” refers to a combination of dosage forms, as well as to a single dosage form, and the like. In some embodiments, the term “or” as used herein, including the claims, is used to mean “and/or” unless explicitly indicated to refer to alternatives only or the alternatives are mutually exclusive.

In some embodiments, numbers expressing quantities of ingredients, properties such as molecular weight, reaction conditions, and so forth, used to describe and claim some embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. In some embodiments, “about” refers to a specified value+/−10%.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

As used herein, “anemia” refers to a rare but treatable disorder in which the bone marrow does not produce blood cells, e.g., red blood cells, white blood cells, and/or platelets. In some embodiments, anemia can be characterized by a deficiency or lack of one type of blood cell or can be characterized by a deficiency or lack of all three types of blood cells. While anemia may be caused by a number of factors or causes, e.g., an iron deficiency, aplastic anemia is caused by a problem with the bone marrow, i.e., an inability of the bone marrow to produce or mature blood cells. Aplastic anemia may be inherited, or it may be acquired after birth, e.g., as a result of exposure to radiation, chemotherapy, toxic chemicals, some drugs, or infection.

As used herein, “blood cell” refers to a cell type produced by the bone marrow. A blood cell may include, but is not limited to, a red blood cell, a white blood cell, and/or a platelet.

As used herein, “bone marrow failure” or “BMF” refers to a condition in a patient in which the bone marrow fails to produce enough cells of one or more major hematopoietic lineages, e.g., red blood cells, and/or platelets, and/or white blood cells, leading to diminished or absent hematopoietic precursors in the bone marrow, a loss of functional blood cells, and attendant cytopenias. All BMF syndromes ultimately arise because of a defect or an insult to hematopoietic stem cells (HSCs). In some embodiments, BMF may be referred to interchangeably as a BMF syndrome.

As used herein, “BMF syndrome” can include, but is not limited to, a condition characterized Fanconi anemia (FA), dyskeratosis congenita (DC), Shwachman-Diamond syndrome (SDS), congenital amegakaryocytic thrombocytopenia (CAMT), Diamond-Blackfan anemia (BDA), and reticular dysgenesis (RD), as well as GATA2-related disorders and SAMD9/SAMD9L-related disorders. In some embodiments, less common disorders that share features with the inherited BMF syndromes listed are included, for example, disorders having short telomerase or shortened telomeres.

As used herein, a “condition characterized by reduced numbers of HSCs” may be permanent or temporary. In some embodiments, a subject with such a condition will have HSCs reduced by >50% as compared to a healthy subject. Examples of such conditions are described herein and include BMF.

As used herein, “co-administration” refers to the combined administration of one or more drugs with another. In some embodiments, both drugs are administered at the same time. Co-administration may also refer to any particular time period of administration of either drug, or both drugs. For example, as described herein, a drug may be administered hours or days before administration of another drug and still be considered to have been co-administered. In some embodiments, co-administration may refer to any time of administration of either drug such that both drugs are present in the body of a patient at the same. In some embodiments, either drug may be administered before or after the other, so long as they are both present within the patient for a sufficient amount of time that the patient received the intended clinical or pharmacological benefits.

As used herein, a dosage unit form or “fixed dose” as used herein refers to physically discrete units suited as unitary dosages for the patients to be treated; each unit contains a predetermined quantity of an LSD1 inhibitor calculated to produce the desired therapeutic effect in association with the required pharmaceutical carrier and, optionally, in association with another agent. Single or multiple dosages may be given. Alternatively, or in addition, the LSD1 inhibitor, or composition(s) comprising these, may be administered via continuous infusion as appropriate.

A pharmaceutical composition(s) comprising an LSD1 inhibitor as described herein may include a “therapeutically effective amount” of the LSD1 inhibitor as described herein. The term “therapeutically effective amount,” “pharmacologically effective dose,” “pharmacologically effective amount,” or simply “effective amount” may be used interchangeably and refers to that amount of an agent effective to produce the intended pharmacological, therapeutic or preventive result, e.g., amelioration of BMF or a BMF syndrome, or associated symptoms thereof in a patient. e.g., an increase in production or maturation of hematopoietic cells, e.g., red blood cells, platelets, and/or white blood cells. The pharmacologically effective amount results in the amelioration of one or more symptoms of a disorder (e.g., BMF or a BMF syndrome), or prevents the advancement of a disorder, or causes the regression of the disorder, or prevents the disorder. Such effective amounts can be determined based on the effect of the administered agent, e.g., an LSD1 inhibitor, or the combinatorial effect of agents if more than one agent is used, e.g., an LSD1 inhibitor followed by a second drug regimen described herein. A therapeutically effective amount of an agent may also vary according to factors such as the disease stage, state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects. In some examples, an “effective amount” is one that treats (including prophylaxis) one or more symptoms and/or underlying causes of BMF or a BMF syndrome. In some examples, an effective amount is a therapeutically effective amount. In some examples, an effective amount is an amount that prevents one or more signs or symptoms of a particular disease or condition from developing.

As used herein, “gene expression” or “expression” refers to the process of gene transcription, translation, and post-translational modification.

As used herein, a “gene expression profile” refers to a specific set of genes transcribed and/or translated in a cell at a certain time or in response to a certain drug. In some embodiments, a biological sample is obtained from a patient before administration of an LSD1 inhibitor such as bomedemstat in order to establish a baseline of gene expression before treatment for BMF or a BMF syndrome. Following administration of bomedemstat, a second biological sample may be obtained from a patient to assess the gene expression of the cells after administration of the LSD1 inhibitor. In this way, the response of BMF or a BMF syndrome in the patient to LSD1 inhibition may be determined. Analysis of gene expression before and/or at certain points of time during or after treatment may reveal the methylation status or expression levels of certain genes in the cells and determine whether the LSD1 inhibitor resulted in the production of hematopoietic cells by the bone marrow.

As used herein. “homeostasis” refers to a state wherein bone marrow niche factors maintain most hematopoietic stem cells (HSCs) in a quiescent state, which is thought to protect HSCs from malignant transformation and malfunction, as proliferating cells are more susceptible to genetic mutations and will become senescent once their turnovers reach their maximum.

As used herein, the “hematopoietic compartment” means all bloods cells in the bone marrow, including mature blood cells, immature blood cells, progenitors, and stem cells. Recovery of the hematopoietic compartment refers to a change in a patient with, e.g., a condition characterized by reduced numbers of hematopoietic stem cells (HSCs), bone marrow failure or a BMF syndrome, or the like, to a state wherein the total number of cells in the BM, and/or the percent of all mature blood cell lineages, approximates, approaches, or is in the same range as that which would be observed in a healthy patient.

As used herein, “immunosuppression” as a treatment option for BMF may include, but is not limited to, standard immunosuppressive treatments such as antithymocyte globulin (ATG), cyclosporine, and/or steroids. In some embodiments, approximately 60 percent of patients respond to immunosuppression, often achieving long-term control of their disease. In some embodiments, immunosuppression may be combined with administration of an LSD1 inhibitor.

As used herein, the term “increase” in reference to HSCs means to increase in number by cellular division and is synonymous with the term “expand.”

As used herein, a “long-term hematopoietic stem cell” or “LT-HSC” refers to a hematopoietic stem cell that is capable of self-renewal. In some embodiments, LT-HSCs have the ability to reestablish or replenish a population of hematopoietic stem cells in the bone marrow, which are able to differentiate into blood cells, e.g., red blood cells, platelets, and/or white blood cells. In some embodiments increasing the population of LT-HSCs provides a treatment for BMF or a BMF syndrome. LT-HSCs can be identified by the expression of specific hematopoietic stem cell markers. In some embodiments, the presence of hematopoietic markers CD34 and CD90 are used to identify LT-HSCs.

As used herein, an “LSD1 inhibitor” refers to a compound or drug that inhibits the activity or expression of LSD1, a nuclear protein with histone demethylase activity. An LSD1 inhibitor may be any compound or drug useful for inhibiting LSD1, such as including, but not limited to, tranylcypromine (TCP), ORY-1001 (iadademstat), IMG-7289 (bomedemstat), INCB059872, OG-L002, ORI-2001 (vafidemstat), seclidemstat (SP-2577), GSK-2879552 2HCl, GSK-LSD1 2HCl, SP2509, Compound 1, Compound 2, Compound 4, CC-90011 besylate, T3775440 HCl, and vafidemstat. LSD1 inhibitors can be classified as reversible or irreversible. In some embodiments, a reversible LSD1 inhibitor may include, but is not limited to, Compound 1, Compound 2, Compound 4, CC-90011 and SP-2577. In some embodiments, an irreversible LSD1 inhibitor may include, but is not limited to, tranylcypromine (TCP), ORY-1001 (iadademstat), GSK-2879552, IMG-7289 (bomedemstat), INCB059872, and ORI-2001 (vafidemstat). Additional LSD1 inhibitors are disclosed herein.

As used herein, the term “near-ablative cancer therapy” means a therapy intended destroy all or nearly all cancerous cells, e.g., by targeting actively dividing cells and/or damaging DNA. Such therapies include radiation therapy and chemotherapy for cancer.

As used herein, the term “niche,” when referring to hematopoietic stem cells or bone marrow, means a specific stem-cell-fate-maintaining microenvironment in which stem cells are housed and maintained by allowing self-renewal in the absence of differentiation. Bone-marrow HSCs and their niches are thought to be either located near the endosteal lining of the bone-marrow cavities (the endosteal niche) or are in close contact to the endothelium of the sinusoids (the vascular niche).

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration or comparable foreign regulatory agencies.

“Pharmacologically active” (or simply “active”) as in a “pharmacologically active” (or “active”) derivative or analog, refers to a derivative or analog having the same type of pharmacological activity as the parent compound and approximately equivalent in degree. Some pharmacologically active derivatives may have improved pharmacological activity.

The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt.

As used herein, an “insult” to the bone marrow includes injury to bone marrow cells such as HSCs from sources such as irradiation and chemical toxicity.

As used herein, a “quiescent cell” refers to a cell in GO stage of the cell division cycle, in which the cell does not divide but retains the ability to re-enter cell proliferation. Some cells remain in a quiescent state and are thus inactive. In some embodiments, a quiescent cell is a hematopoietic stem cell. As described herein, administration of an LSD1 inhibitor to a patient may alter or modify the gene expression profile or program of a cell such that the cell becomes active and begins to proliferate. In some embodiments, administration of the LSD1 inhibitor results in the hematopoietic stem cells in the bone marrow entering the cell division cycle, thereby increasing the number of hematopoietic stem cells in the bone marrow. In some embodiments, increasing the number of hematopoietic stem cells in the bone marrow results in amelioration of symptoms associated with BMF or a BMF syndrome. In some embodiments, the hematopoietic stem cells that are increased are long-term hematopoietic stem cells (LT-HSCs), which are capable of self-renewal and provide the opportunity to replenish the ability of the bone marrow to produce blood cells, e.g., red blood cells, platelets, and/or white blood cells.

As used herein, “recovery period” refers to the period after administration of a drug treatment or therapeutic intervention. A recovery period may also refer to the period after administration of a drug and before administration of a second drug treatment. For example, in some embodiments, a recovery period may refer to the period after administration of a chemotherapeutic drug regimen and before administration of an LSD1 inhibitor. As described herein, administration of the LSD1 inhibitor after administration of a chemotherapeutic drug or regimen, or after a recovery period as described herein, may enhance regeneration of bone marrow cells, such as bone marrow stem cells, resulting in amelioration of BMF or symptoms thereof.

As used herein. “reducing” refers to a lowering or lessening, such as reducing BMF or symptoms associated with BMF or a BMF syndrome. In some embodiments, administration of an LSD1 inhibitor as described herein may result in “reduced” or lessened BMF or a BMF syndrome or associated symptoms in the patient compared to a patient not having been administered such drugs. “Reducing” may also refer to a reduction in disease symptoms as a result of a treatment as described herein, either alone, or co-administered with another drug.

As used herein, “reprogramming” refers to an alteration or modification in the gene expression of a hematopoietic stem cell or hematopoietic cell in response to an LSD1 inhibitor, such that the cell may divide and produce more hematopoietic cells or cells of hematopoietic lineage, e.g., red blood cells, platelets, and/or white blood cells. In some embodiments, administration of an LSD1 inhibitor may enable bone marrow cells to mature into functional hematopoietic cells or cells of hematopoietic lineage, e.g., red blood cells, platelets, and/or white blood cells. In some embodiments, the reprogramming of a hematopoietic stem cell or hematopoietic cell in response to the administration of an LSD1 inhibitor is the result of the alteration or modification of the histone demethylase activity of LSD1 in the cell. In some embodiments, administration of an LSD1 inhibitor may push or induce cells to enter the cell division cycle, representing a change in the gene expression profile of that cell by LSD1 inhibition, rendering them active and able to divide and reproduce more hematopoietic cells.

As used herein, “subject” or “individual” or “patient” refers to any patient for whom or which therapy is desired, and generally refers to the recipient of the therapy. A “subject” or “patient” refers to any animal classified as a mammal, e.g., human and non-human mammals. Examples of non-human animals include dogs, cats, cattle, horses, sheep, pigs, goats, rabbits, etc. Unless otherwise noted, the terms “patient” or “subject” are used herein interchangeably. In some embodiments, a subject amenable for therapeutic applications may be a primate, e.g., human and non-human primates.

The terms “treating” and “treatment” or “alleviating” as used herein refer to reduction or lessening in severity and/or frequency of symptoms, elimination of symptoms and/or underlying cause, and improvement or remediation of damage. In certain aspects, the term “treating” and “treatment” as used herein refer to the prevention of the occurrence or recurrence of symptoms. In other aspects, the term “treating” and “treatment” as used herein refer to the prevention of the underlying cause of symptoms associated with a disease or condition, such as BMF or a BMF syndrome. The phrase “administering to a patient” refers to the process of introducing a composition or drug into the patient via an art-recognized means of introduction. “Treating” or “alleviating” also includes the administration of compounds or agents to a subject to prevent or delay the onset of the symptoms, complications, or biochemical indicia of a disease (e.g., BMF or a BMF syndrome), alleviating the symptoms or arresting or inhibiting further development of the disease, condition, or disorder. Subjects in need of treatment include those already suffering from the disease or condition, those previously suffering from the disease or condition and at risk of recurrence, as well as those being at risk of developing the disease or condition. Treatment may be prophylactic (to prevent or delay the onset of the disease or condition, or to prevent the manifestation of clinical or subclinical symptoms thereof) or therapeutic suppression, or alleviation of symptoms after the manifestation of the disease or condition.

All methods described herein can be performed in any suitable order unless otherwise indicated herein or otherwise clearly contradicted by context. The use of any and all examples, or exemplary language (e.g., “such as”) provided with respect to some embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or d from, a group for reasons of convenience or patentability.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Examples of embodiments of the present disclosure are provided in the following examples. The following examples are presented only by way of illustration and to assist one of ordinary skill in using the disclosure. The examples are not intended in any way to limit the scope of the disclosure.

### Example 1

As described herein, an LSD1 inhibitor, such as bomedemstat, is administered orally once daily to a patient diagnosed as having BMF or a BMF syndrome for a period of 7 days at a dose of 3 mg/kg to 6 mg/kg, or a total daily dose of 10 mg to 700 mg.

Bomedemstat is an orally available small molecule discovered and developed by Imago BioSciences that inhibits lysine-specific demethylase 1 (LSD1 or KDM1A). It is expected that the administration of bomedemstat will result in bone marrow cells dividing and maturing, and the number of HSCs increasing, to reduce and/or treat a condition characterized by reduced numbers of HSCs, such as BMF or a BMF syndrome or associated symptoms thereof, and/or to enhance recovery from or improve tolerance of a near-ablative cancer therapy, and/or to reduce the damage to or accelerate the recovery of the hematopoietic compartment after a physical insult thereto, in a patient in need thereof. LSD1 inhibitors may be administered in vivo or may be used to treat patient or donor HSCs ex vivo to produce an expanded population of HSCs which may then be infused into the patient.

### Example 2

As described herein, an LSD1 inhibitor, such as bomedemstat, may be administered orally once daily to a patient diagnosed as having BMF or a BMF syndrome for a period of 5 days at a dose of 3 mg/kg to 6 mg/kg, or a total daily dose of 10 mg to 700 mg.

### Example 3

Blood or bone marrow samples may be obtained using methods known in the art, e.g., the composition of the various blood cells analyzed using a fluorescently activated cell sorter (FACS) to enumerate the proportion of stem/progenitor cells. For example, CD34⁺CD38⁻ cells, CD34⁺EPCR⁺CD45RA⁻CD38⁻ cells, or another target population of HSCs can be identified and quantified at baseline. Then, an LSD1 inhibitor, such as bomedemstat is administered orally once daily to a patient diagnosed as having BMF or a BMF syndrome for a period of 7 days at a dose of 3 mg/kg to 6 mg/kg, or a total daily dose of 10 mg to 700 mg.

Following the 7 days of treatment with bomedemstat, a second blood or bone marrow sample is obtained from the patient and a FACS analysis is performed using methods known in the art, in order to assay the change in the composition of cells, e.g., and absolute or relative increase in the HSPC fraction of bone marrow-derived or peripheral blood cells.

It is expected that the administration of an LSD1 inhibitor, such as bomedemstat, will result in stem and progenitor bone marrow cells dividing to lessen the onset of frank BMF or a BMF syndrome, or associated signs and symptoms in the patient.

### Example 4

The effect of LSD1 inhibitors on HSCPs was investigated in in vivo studies.

Animals. Healthy animals with an intact immune system were used for all experiments. All animals were drug and test naïve and not involved in previous procedures. Animals were maintained on a 12 hour light-dark cycle with access to water and standard chow ad libitum. CRADL animal care staff conducted routine husbandry procedures and provided daily care and monitoring of all animals housed in CRADL's animal facilities. 8-week old female C57/BL6 (C57BL/6NCrl) mice were purchased from Charles River. All mice were acclimated for at least 48 hours upon arrival. Mice were assigned to treatment groups based on complete blood counts to achieve congruent profiles, body weight was recorded for dose adjustments, and ear tagged for identification. Investigators were not blinded to the identity of mice or samples. No statistical methods were utilized to determine sample size. The experiments described in this study were designed to use the minimum number of animals required.

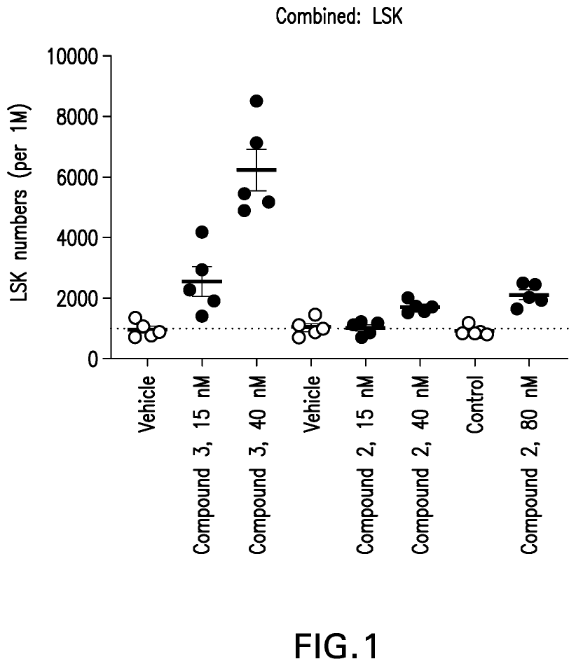

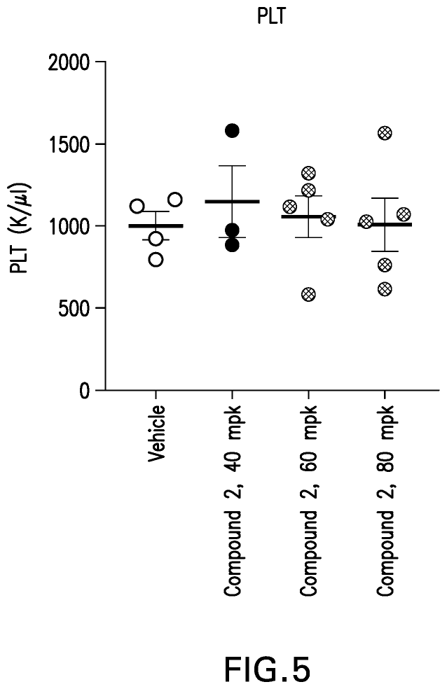

Dosing. To assess the effect of different LSD1 inhibitors on hematopoietic stem and progenitor cells (HSPCs), wild-type mice were administered with Compound 3, Compound 2, or vehicle once daily for 7 days via oral gavage. Administered doses were as indicated in FIGS. 1-5 (Compound 3: 15 and 40 mg/kg, Compound 2, 15, 40, 60, and/or 80 mg/kg).

Drug preparation. As an example of formulation calculations, a correction factor (compound and lot dependent) of 1.5 will be used (e.g., one lot of Compound 2 actual correction factor 1.67; one lot of Compound 3 actual correction factor 1.69). Vehicle is 30% PEG400 (Millipore Sigma cat #8074850058), 5% Cremophor EL (Med Chem Express cat #HY-Y189) in sterile Milli-Q water.

Compound 3 or Compound 2 (salt) at a dose of 15 mg/kg dose was prepared by weighing out 0.3 mg×1.5 (example correction factor)=0.45 mg per mouse in 200 μl vehicle solution. Each mouse of 20 g received 200 μl of compound in vehicle solution (volume was corrected for body weight of each mouse at time of dosing). Calculations were scaled appropriately for higher doses. Compound was completely dissolved by swirling and gently pipetting in vehicle. Aliquots were stored for daily usage at 4 C in the dark. On the day of dosing, formulations and vehicle were removed from 4 C and allowed to warm to RT for at least 20 minutes before dosing. Formulations were swirled to achieve a homogenous solution prior to administration for 7 days, once daily, via oral gavage.

Processing of bones and blood for analysis. 24 hours after the last dose was administered, blood was collected via cheek bleed and mice were subsequently sacrificed via CO2 asphyxiation. Bones were separated from the mice, muscle and tissue removed, and bone marrow cells were isolated from mouse femora and tibiae by centrifugation. Red blood cells (RBC) were lysed by adding cold ACK lysis buffer (Thermo Fisher cat #A1049201) and incubating bone marrow cells (or 20-30 ml of peripheral blood) on ice for 10 minutes. Following RBC lysis, samples were topped up with an appropriate volume of FACS buffer (PBS+2% BSA) and centrifuged at 3000 rpm for 5 minutes. Cell pellet was resuspended in FACS buffer and filtered through a 70 mM filter to remove aggregated cell clumps and debris. Cells in the filtered suspension were counted and ˜2-3×10⁶ cells removed for cell staining.

Complete blood counts. Complete blood counts from the peripheral blood sample collected at end of study were measured at IDEXX BioAnalytics.

Flow Cytometry Analysis. For flow staining, @ 2-3×10e6 cells were centrifuged at 400×g, 5 minutes, 4 C in 1.5 ml centrifuge tubes. Supernatant was aspirated and cells were resuspended in 100 μl of staining cock-tail per tube. Tubes were incubated on ice for 30 minutes in the dark. 1 ml of FACS buffer (PBS+2% FBS) was added to each tube and then centrifuged at 400×g, 5 minutes, 4° C. Supernatant was aspirated, and cells were resuspended at 300 μl per tube in 7-AAD solution (5 μl/ml of FACS Buffer). Samples were filtered through a 70 mM filter into 5 ml FACS tubes and analyzed on a SONY SA3800 Analyzer.

| | Antigen | Color | μL/100 μL | Catalog | Company |
|---|---|---|---|---|---|
| Panel LSK/ | Lineage | AF700 | 3 | 133313 | BioLegend |
| Progenitor | Cocktail* | | | | |
| BM | CD117 | BV605 | 2 | 105847 | BioLegend |
| | Sca1 | PECγ7 | 1 | 108113 | BioLegend |
| | CD34 | FITC | 2 | 11-0341-82 | Thermo |
| | CD16/32 | BV421 | 1 | 101332 | BioLegend |
| | CD150 | PECy7 | 2 | 115903 | BioLegend |
| | CD48 | PacBlue | 2 | 103417 | BioLegend |
| | Viability | 7-AAD | 5 μL/mL | 00-6993-50 | Thermo |
| | Stain | | | | |

*= CD3, Gr-1, B220, Ter-119, CD11b

Data were analyzed using Flow Jo Software.

Gating strategies for the populations described were as following:

| Population | Gating strategy |
|---|---|
| HSC (LT-HSC) | Lin-cKIT⁺Sca1⁺⁻, CD150⁺CD48⁻ |
| HPC1 | Lin-cKIT⁺Sca1⁺⁻, CD150⁻CD48⁺ |
| HPC2 | Lin-cKIT⁺Sca1⁺⁻, CD150⁺CD48⁺ |
| MPP | Lin-cKIT⁺Sca1⁺⁻, CD150⁻CD48⁻ |
| LSK | Lin-cKIT⁺Sca1⁺ |
| MP | Lin-cKIT⁺Sca1⁻ |
| CMP | Lin-cKIT⁺Sca1⁻, CD16.32^{mid}, CD34^{mid} |
| MEP | Lin-cKIT⁺Sca1⁻, CD16.32⁻, CD34⁻ |
| GMP | Lin-cKIT⁺Sca1⁻, CD16.32⁺, CD34⁺ |

| Antigen | Clone |
|---|---|
| CD150 | TC15-12F12.2 |
| CD48 | HM48-1 |
| CD16/32 | 93 |
| CD34 | RAM34 |
| CD117 | 2B8 |
| Sca1 | D7 |
| Lineage cocktail | CD3 clone 17A2, Gr-1 clone RB6-8C5, |
| CD3, Gr-1, Ter119, | B220 clone RA3-6B2, Ter119 clone |
| B220, CD11b | Ter-119, CD11b clone M1/70 |
| Viability dye | 7-AAD |

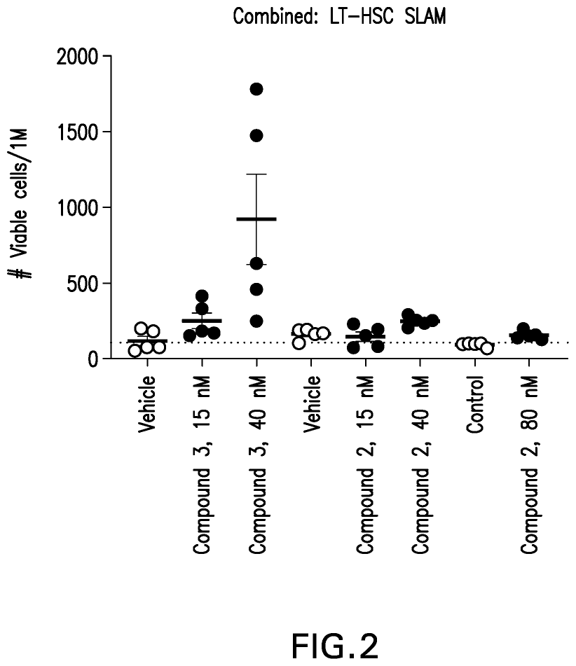

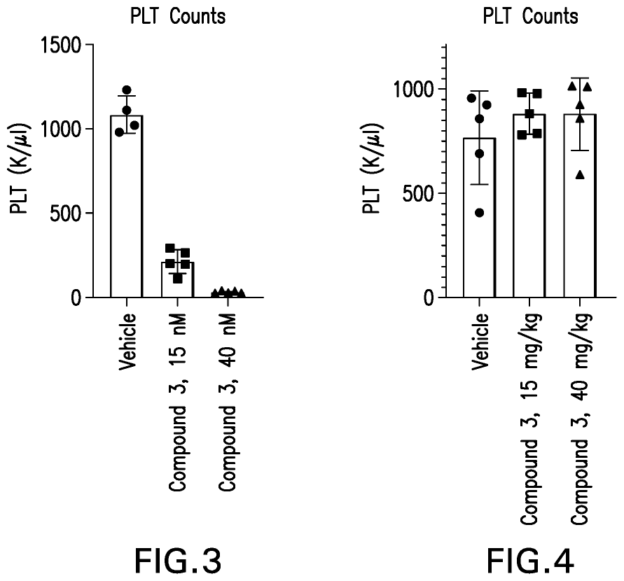

Results are shown below in FIGS. 1-5. FIGS. 1 and 2 show an increase in the number of LSK cells and LT-HSC cells, respectively, as a result of LSD1 treatment. FIGS. 3-5, collectively, show that platelet count is not necessarily affected by LSD1 treatment.

### Example 5

The effect of LSD1 inhibitors on HSCPs was investigated in ex vivo studies.

HSC Proliferation Assays. HSC ex vivo assays were carried out using mPB CD34+ cells (Fred Hutchinson Cancer Research Center (FHCRC), Seattle, WA). Cells were thawed according to the protocol supplied by FHCRC and resuspended in HSC Proliferation Media (StemSpan SFEM II+StemSpan CC100 cocktail from Stem Cell Technologies+Penicillin-Streptomycin from Thermo Fisher) A viable cell count was determined by trypan blue staining. Cells were plated into a 96 well tissue culture plate a 5,000 cells per well in 100 μl HSC Proliferation Media. Stock test compounds (25 mM in DMSO) were diluted into HSC Proliferation media at a 2× final concentration and added at 100 μl per well. Final volume in each well was 200 μl with DMSO concentration per well not exceeding 0.02%. Each treatment was tested in triplicate. Final test concentrations tested are shown in the chart below. Plates were incubated for 6 days in a tissue culture incubator, 37 C, 5% CO₂. On the 6^{th} day, cell counts per well were collected and cells were analysed by flow cytometry.

| | Final concentration |
|---|---|
| Compound | (nM) |
| UM-171 | 35 |
| Compound 3 | 30, 100, 300 |
| Compound 2 | 30, 100, 300 |
| Compound 1 | 3, 10, 30 |
| Compound 4 | 10, 30, 100 |

Flow Cytometry Analysis. For flow staining, the 96 well plate was centrifuged at 500×g, 5 minutes, 4° C. Supernatant was removed, and 150 μl cold FACS buffer (PBS+2% FBS) was added to each well. Plate was centrifuged at 400×g, 5 minutes, 4° C., and supernatant was removed. Cells were resuspended in 50 μl of staining cocktail per well. Plate was incubated on ice for 30 minutes in the dark. 15 μl of FACS buffer was added to each well, and the plate was centrifuged at 500×g, 5 minutes, 4 C. Supernatant was removed, and cells were resuspended in 200 μl per well 7-AAD solution (5 μl/ml in FACS Buffer). Samples were analysed on a SONY SA3800 Analyzer. HSC Proliferation staining panel shown below:

| | Antigen | Color | μl/100 μl | Catalog # | Company | Clone |
|---|---|---|---|---|---|---|
| Panel HSC | CD34 | PacBlue | 2 | 343511 | BioLegend | 581 |
| Proliferation | CD38 | PE Cy7 | 1 | 25-0389-42 | Thermo Fisher | HIT2 |
| mPB | CD90 | BV605 | 3 | 328128 | BioLegend | 5E10 |
| CD34⁺ | CD45RA | FITC | 2.5 | 11-0458-42 | Thermo Fisher | HI101 |
| | ITGA3 | BV421 | 1 | 744516 | BD BioSciences | C3 II.1 |
| | EPCR | PE | 1 | 351904 | BioLegend | RCR-401 |
| | Viability | 7-AAD | 5 μl/ml | 00-6993-50 | Thermo | |
| | Stain | | | | | |

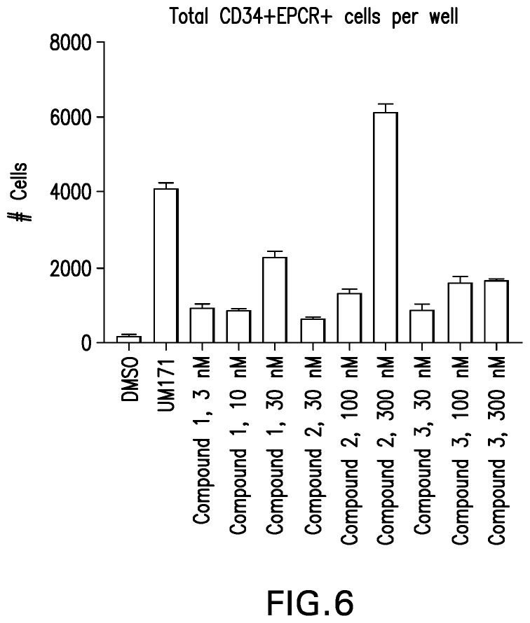

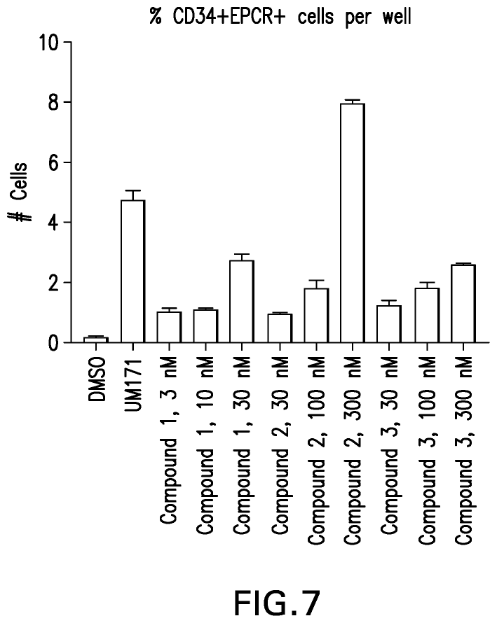

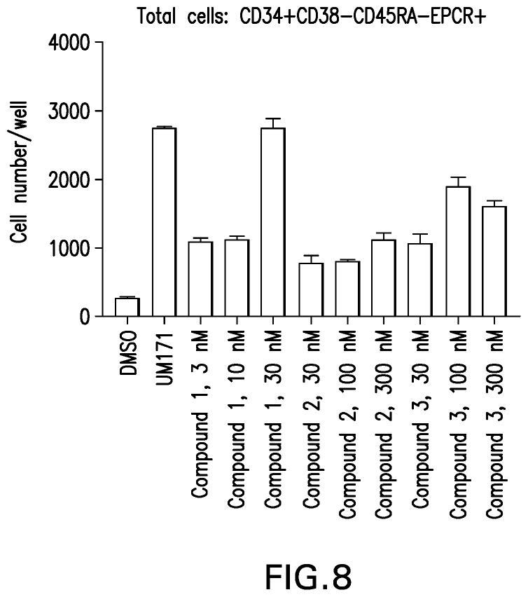

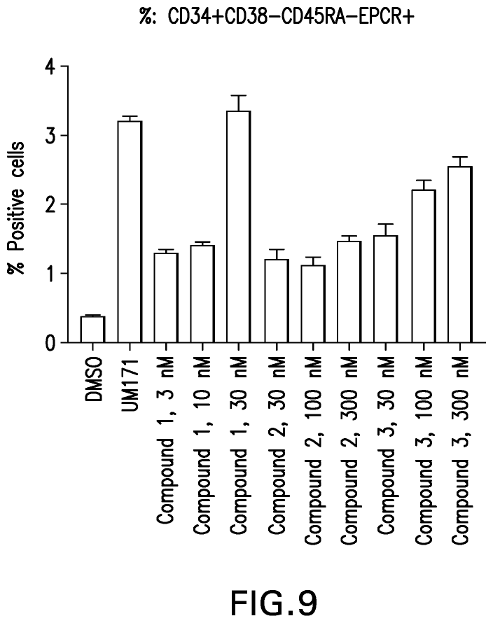

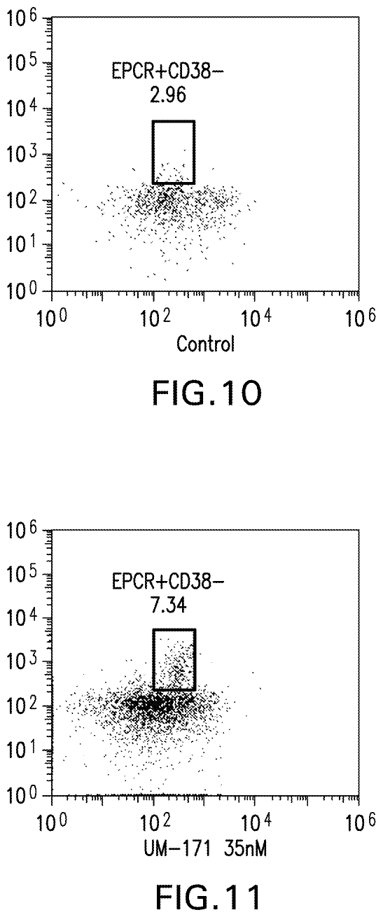

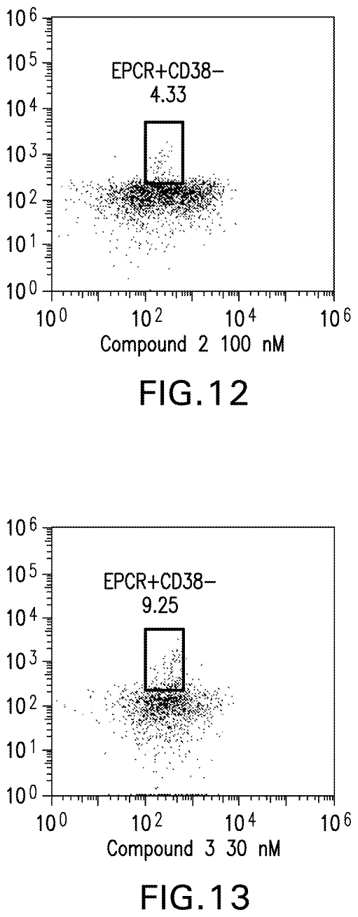

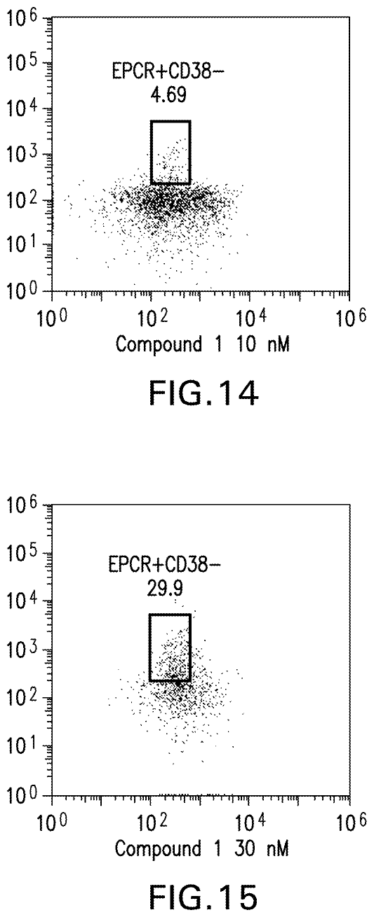

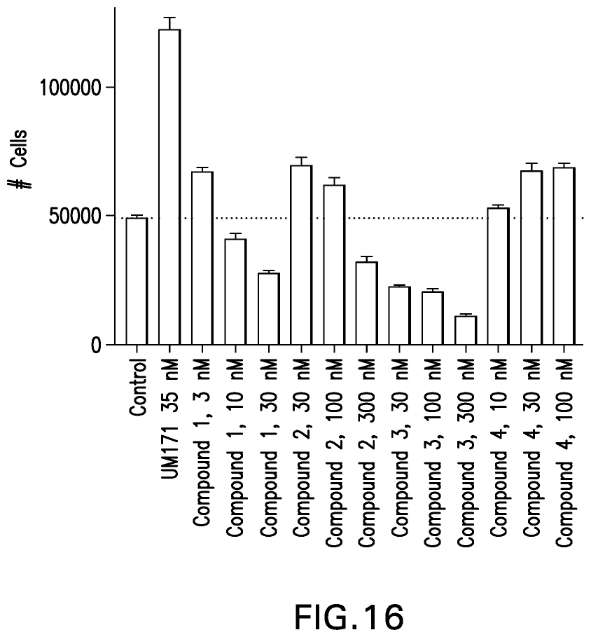

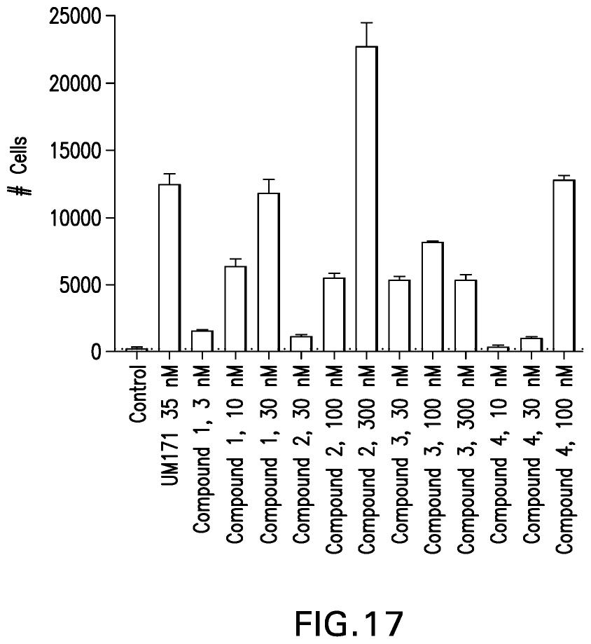

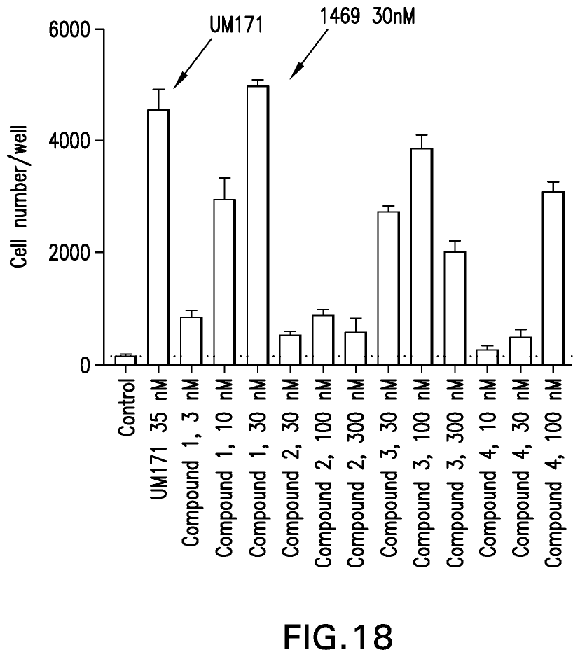

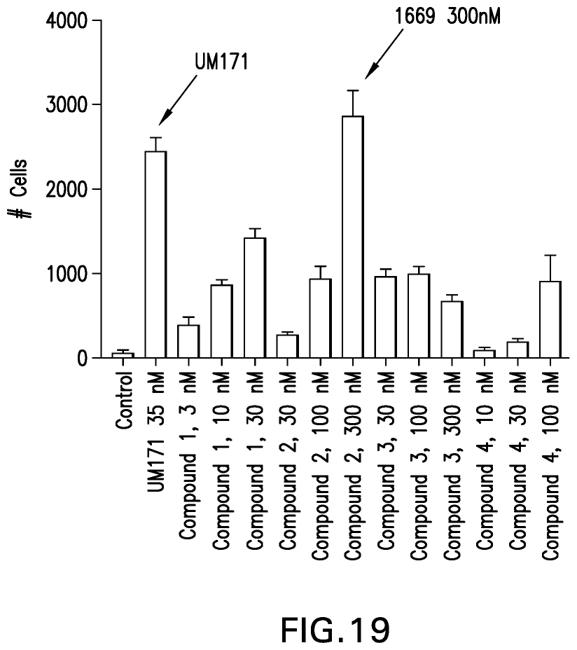

Results are shown below in FIGS. 6-19. As shown in FIGS. 6-9, LSD1 inhibition results in increase in increase in both the total number and the percent of CD34⁺EPCR⁺ and CD34⁺EPCR⁺CD38⁻CD45RA⁻ HSCs. As shown in FIGS. 10-15, LSD1 inhibition results in increase in the percent of CD34⁺EPCR⁺CD38⁻CD45RA⁻ HSCs. As shown in FIGS. 16-19, LSD1 inhibition results in increase in total number per well of CD34+ cells (FIG. 16), CD34⁺EPCR⁺ HSCs (FIG. 17), CD34+EPCR+CD38−CD45RA− HSCs (FIG. 18), and CD34+EPCR+CD90+CD38− HSCs (FIG. 19).

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

1. A method of increasing the numbers of hematopoietic stem cells in a patient in need thereof comprising i) administering an LSD1 inhibitor to the patient in an amount and/or for a period of time sufficient to result in an in vivo expansion of a population of hematopoietic stem cells; or ii) contacting hematopoietic stem cells harvested from a patient or a suitable donor with an LSD1 inhibitor ex vivo, in an amount and/or for a period of time sufficient to result in an expansion of a population of hematopoietic stem cells, then administering to the patient a therapeutically effective amount of the expanded population of hematopoietic stem cells.

2. The method of claim 1 wherein the patient has a condition characterized by reduced numbers of hematopoietic stem cells.

3. A method of treating a condition characterized by reduced numbers of hematopoietic stem cells in a patient in need thereof, comprising i) administering an LSD1 inhibitor to the patient in an amount and/or for a period of time sufficient to result in an in vivo expansion of a population of hematopoietic stem cells; or ii) contacting hematopoietic stem cells harvested from a patient or a suitable donor with an LSD1 inhibitor ex vivo, in an amount and/or for a period of time sufficient to result in an expansion of a population of hematopoietic stem cells, then administering to the patient a therapeutically effective amount of the expanded population of hematopoietic stem cells.

4. A method of enhancing recovery from a near-ablative cancer therapy comprising, subsequent to such therapy, i) administering an LSD1 inhibitor to the patient in an amount and/or for a period of time sufficient to result in an in vivo expansion of a population of hematopoietic stem cells; or ii) contacting hematopoietic stem cells harvested from a patient or a suitable donor with an LSD1 inhibitor ex vivo, in an amount and/or for a period of time sufficient to result in an expansion of a population of hematopoietic stem cells, then administering to the patient a therapeutically effective amount of the expanded population of hematopoietic stem cells.

5. A method of improving tolerance of a near-ablative cancer therapy comprising, prior to such therapy, i) administering an LSD1 inhibitor to the patient in an amount and/or for a period of time sufficient to result in an in vivo expansion of a population of hematopoietic stem cells; or ii) contacting hematopoietic stem cells harvested from a patient or a suitable donor with an LSD1 inhibitor ex vivo, in an amount and/or for a period of time sufficient to result in an expansion of a population of hematopoietic stem cells, then administering to the patient a therapeutically effective amount of the expanded population of hematopoietic stem cells.

6. The method of claim 5, wherein the near-ablative cancer therapy is administered after a period of time after administration of the LSD1 inhibitor sufficient for cells to re-enter the quiescent phase.

7. The method of claim 6, wherein the period of time sufficient for cells to enter the quiescent phase is between about one and about two weeks.

8. The method of 4, wherein the near-ablative cancer therapy is chosen from therapeutic irradiation of cancer cells and cytotoxic chemotherapy of cancer cells.

9. A method of reducing the damage to, or accelerating the recovery of, the hematopoietic compartment after a physical insult thereto in a patient in need thereof, comprising i) administering an LSD1 inhibitor to the patient in an amount and/or for a period of time sufficient to result in an in vivo expansion of a population of hematopoietic stem cells; or ii) contacting hematopoietic stem cells harvested from a patient or a suitable donor with an LSD1 inhibitor ex vivo, in an amount and/or for a period of time sufficient to result in an expansion of a population of hematopoietic stem cells, then administering to the patient a therapeutically effective amount of the expanded population of hematopoietic stem cells.

10. The method of claim 7, wherein the physical insult is a result of irradiation or chemical toxicity.

11. The method of claim 1, wherein administration of the LSD1 inhibitor results in proliferation of the hematopoietic stem cells in the bone marrow.

12. The method of claim 1, wherein administration of the LSD1 inhibitor reduces a proportion of quiescent cells in the hematopoietic stem cell population.

13. The method of claim 1, wherein administration of the LSD1 inhibitor results in the hematopoietic stem cells entering the cell division cycle, thereby increasing the number of hematopoietic stem cells in the bone marrow and in the peripheral circulation.

14-19. (canceled)

20. The method of claim 1, wherein the numbers of hematopoietic stem cells are reduced by 50% prior to administration of the LSD1 inhibitor.

21. The method of claim 20, wherein the reduced numbers of hematopoietic stem cells classify the patient as having bone marrow failure.

22. The method of claim 21, wherein reduced numbers of peripheral blood cells, i.e., cytopenia, anemia, thrombocytopenia, lymphopenia, or neutropenia classify the patient as having bone marrow failure.

23. The method of claim 1, wherein the condition characterized by reduced numbers of hematopoietic stem cells is chosen from a bone marrow failure syndrome, a myeloproliferative neoplasm, and a myelodysplastic syndrome.

24-33. (canceled)

34. The method of claim 1, wherein the condition characterized by reduced numbers of hematopoietic stem cells is an autoimmune condition causing aplastic anemia.

35. The method of claim 1, wherein the condition characterized by reduced numbers of hematopoietic stem cells is the result of a mutation in a lineage cell.

36. The method of claim 35, wherein the lineage cell is a progenitor cell of the erythroid lineage, megakaryocytic lineage, or granulo-monocytic lineage.

37. The method of claim 35, wherein the lineage cell is chosen from common myeloid progenitor, erythroid progenitor cell, megakaryocytic progenitor cell, a granulocyte-monocyte progenitor, or a megakaryocytic-erythroid progenitor cell.

38. The method of claim 35, wherein the mutation in a lineage cell leads to altered numbers of, malformations in, or impaired function of one or more cells chosen from granulocytes, megakaryocytes, erythrocytes, neutrophils, basophils, eosinophils, monocytes, and dendritic cells.

39-49. (canceled)

50. The method of claim 1, wherein the dose of the LSD1 inhibitor is from about 0.25 mg/kg to about 7 mg/kg.

51. The method of claim 1, wherein the dose of the LSD1 inhibitor is from about 10 mg to about 700 mg.

52-66. (canceled)

67. The method of claim 1, wherein the LSD1 inhibitor is 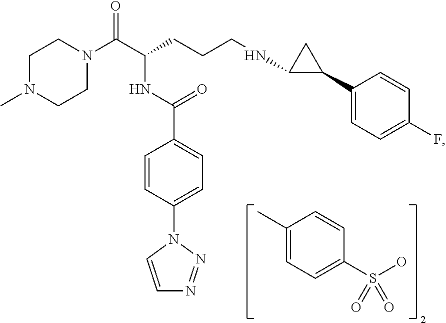
or a polymorph or solvate thereof.

68. (canceled)
